(19) United States
(12) Patent Application Publication
(10) Pub. No.: US 2005/0069963 A1
(43) Pub. Date: Mar. 31, 2005

(54) Multifactorial assay for cancer detection

(76) Inventors: Anna E. Lokshin, Pittsburgh, PA (US); Elieser Gorelik, Pittsburgh, PA (US)

Correspondence Address: KIRKPATRICK & LOCKHART NICHOLSON GRAHAM LLP, 535 SMITHFIELD STREET, PITTSBURGH, PA, 15222, US

(21) Appl. No.: 10/918,727

(22) Filed: Aug. 13, 2004

Related U.S. Application Data
(60) Provisional application No. 60/495,547, filed on Aug. 15, 2003.

(51) Int. Cl. G01N 33/574; A61K 38/00

(52) U.S. Cl. 435/7.23; 514/2

(57) ABSTRACT

Provided are methods for the rapid detection of ovarian cancer. The methods employ a multiplex immunoassay to detect levels of two or more of the markers EGF, G-CSF, IL-6, IL-8, CA-125, VEGF, MCP-1, anti-IL6, anti-IL8, anti CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu in a sample of the patient's blood, where the presence of abnormal levels of two or more of the markers indicates the presence of ovarian cancer in the patient. An array also is provided to quantitate levels of these markers in a patient's blood. Also provided is a method of predicting onset of clinical ovarian cancer comprising determining the change in concentration over time of two or more of anti-Her2/neu, anti-MUC-1, anti-c-myc, anti-p53, anti-CA-125, anti-CEA, anti-CA 72-4, anti-PDGFRα, IFNγ, IL-6, IL-10, TNFα, MIP-1α, MIP-1β, EGFR and Her2/neu in a patient's blood.

### CROSS REFERENCE TO RELATED APPLICATIONS

This application claims priority under 35 U.S.C. §119(e) to U.S. Provisional Patent Application No. 60/495,547, filed Aug. 15, 2003, which is incorporated herein by reference in its entirety.

### BACKGROUND

1. Field of the Invention

Methods and reagents for a multifactorial assay for the rapid, early detection of cancer.

2. Description of the Related Art

Ovarian cancer represents the third most frequent cancer of the female genital tract. The majority of early-stage cancers are asymptomatic, and over three-quarters of the diagnoses are made at a time when the disease has already established regional or distant metastases. Despite aggressive cytoreductive surgery and platinum-based chemotherapy, the 5-year survival for patients with clinically advanced ovarian cancer is only 15 to 20 percent, although the cure rate for stage I disease is usually greater than 90 percent (Holschneider, C. H. and J. S. Berek, Ovarian cancer: epidemiology, biology, and prognostic factors. Semin Surg Oncol, 2000. 19(1): p. 3 -10). These statistics provide the primary rationale to improve ovarian cancer screening and early identification.

Epithelial ovarian cancer is so deadly in part because of a lack of effective early detection methods. If detected early, survival is dramatically increased. Current research is now focusing on developing improved ways of evaluating women, particularly those at high risk to develop ovarian cancer. As yet, however, a premalignant lesion has not been identified. Although alterations of several genes, such as c-erb-B2, c-myc, and p53, have been identified in a significant fraction of ovarian cancers, none of these mutations are diagnostic of malignancy or predictive of tumor behavior over time (Veikkola, T., et al., Regulation of angiogenesis via vascular endothelial growth factor receptors. Cancer Res, 2000. 60(2): p. 203-12; Berek, J. S., et al., Serum interleukin-6 levels correlate with disease status in patients with epithelial ovarian cancer. Am J Obstet Gynecol, 1991. 164(4): p. 1038-42; discussion 1042-3; Cooper, B. C., et al., Preoperative serum vascular endothelial growth factor levels: significance in ovarian cancer. Clin Cancer Res, 2002. 8(10): p. 3193-7; and Di Blasio, A. M., et al., Basic fibroblast growth factor and ovarian cancer. J Steroid Biochem Mol Biol, 1995. 53(1-6): p. 375-9). Instead, high-risk women must rely on genetic counseling and testing, as well as measurement of serum CA-125 level and transvaginal ultrasound (Oehler, M. K. and H. Caffier, Prognostic relevance of serum vascular endothelial growth factor in ovarian cancer. Anticancer Res, 2000. 20(6D): p. 5109-12; Santin, A. D., et al., Secretion of vascular endothelial growth factor in ovarian cancer. EurJ Gynaecol Oncol, 1999. 20(3): p. 177-81; and Senger, D. R., et al., Tumor cells secrete a vascular permeability factor that promotes accumulation of ascites fluid. Science, 1983. 219(4587): p. 983-5). However, CA-125 is neither sensitive nor specific for detecting early stage disease. Current recommendations do not favor it for general screening. It is only thought to be robust in monitoring the response or progression of the disease, but not as a diagnostic or prognostic marker (Gadducci, A., et al., Serum preoperative vascular endothelial growth factor (VEGF) in epithelial ovarian cancer: relationship with prognostic variables and clinical outcome. Anticancer Res, 1999. 19(2B): p. 1401-5).

Screening using transvaginal ultrasound, Doppler and morphological indices has shown some encouraging results but, used alone, it currently lacks the specificity required of a screening test for the general population (Karayiannakis, A. J., et al., Clinical significance of preoperative serum vascular endothelial growth factor levels in patients with colorectal cancer and the effect of tumor surgery. Surgery, 2002. 131(5): p. 548-55 and Lee, J. K., et al., Clinical usefulness of serum and plasma vascular endothelial growth factor in cancer patients: which is the optimal specimen? Int J Oncol, 2000. 17(1): p. 149-52). Combinational multimodal screening using tumor markers and ultrasound yields higher sensitivity and specificity. This combination approach is also the most cost-effective potential screening strategy (Karayiannakis et al., 2002 and Lee et al., Int J Oncol, 2000). However, it, too, is of questionable effectiveness in the general population. Thus, there is a critical need to develop additional markers for early detection of disease.

Recently, a novel technology named Surface-Enhanced Laser Desorption/Ionization Time-of-Flight Mass Spectrometry (SELDI-TOF-MS) that combines solid phase protein chromatography and mass spectrometry (reviewed in (Issaq, H. J., et al., The SELDI-TOF MS approach to proteomics: protein profiling and biomarker identification. Biochem Biophys Res Commun, 2002. 292(3): p. 587-92)), has been utilized as a novel approach to biomarker discovery in ovarian cancer. In a recently published landmark study of ovarian cancer patients, the new technique has been utilized for protein profiling of ovarian cancer progression (Petricoin, E. F., et al., Use of proteomic patterns in serum to identify ovarian cancer. Lancet, 2002. 359(9306): p. 572-7). This approach allowed for discriminating serum protein profiles with a positive predictive value of 94% as compared with 34% for CA-125. However, as high as this value is, due to the low the incidence of ovarian cancer in the population likely to be screened, the positive predictive value must be almost 100% to avoid generating a high number of false positives. Thus, additional markers are necessary to provide the required high level of specificity and positivity that are required to utilize this approach for the effective general population screening for ovarian cancer. Additionally, this approach is very expensive and could only be applied to high-risk population.

It is well known that ovarian cancer cells produce various angiogenic factors and stimulate secretion of various cytokines, which can be potentially used as biomarkers. However, each single factor was only weakly associated with early stage disease. It was hypothesized that evaluation of a panel of several angiogenic factors and cytokines in the serum of each individual patient will provide sufficient specificity and sensitivity for diagnostic of early stages ovarian cancer. All previous testing of serum markers of cancer patients was performed using ELISA, which is very expensive and requires a separate kit for each individual cytokine.

### SUMMARY

A method for rapid, early detection of ovarian cancer is provided. The method provides the opportunity to simultaneously test a broad panel of angiogenic factors and repeat such testing at multiple time points with use of only, for example and without limitation, 50 μl of serum or plasma per time point.

A method of assaying for the presence of ovarian cancer in a patient is provided. Also provided is a method for predicting the presence of, or outcome of ovarian cancer in a patient. The methods comprise A method of determining the presence of ovarian cancer in a patient, comprising determining levels of markers in a blood marker panel comprising two or more of EGF (Epidermal Growth Factor), G-CSF (Granulocyte Colony Stimulating Factor), IL-6 (Interleukin 6, with “IL”, as used herein, referring to “interleukin”), IL-8, CA-125 (Cancer Antigen 125), VEGF (Vascular Endothelial Growth Factor), MCP-1 (monocyte chemoattractant protein-1), anti-IL6, anti-IL8, anti-CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu in a sample of the patient's blood, where the presence of two or more of the following conditions indicates the presence of ovarian cancer in the patient: EGF₁₀, G-CSF_{HI}, IL-6_{HI}, IL-8_{HI}, VEGF_{HI}, MCP-1_{LO}, anti-IL-6_{HI}, anti-IL-8_{HI}, anti-CA-125_{HI}, anti-c-myc_{HI}, anti-p53_{HI}, anti-CEA_{HI}, anti-CA 15-3_{HI}, anti-MUC-1_{HI}, anti-survivin_{HI}, anti-bHCG_{HI}, anti-osteopontin_{HI}, anti-Her2/neu_{HI}, anti-Akt1_{HI}, anti-cytokeratin 19_{HI} and anti-PDGF_{HI}, CA-125_{HI}, cytokeratin 19_{HI}, EGFR_{LO}, Her2/neu_{LO}, CEA_{HI}, FasL_{HI}, kallikrein-8_{LO}, ErbB2_{LO} and M-CSF_{LO}. Exemplary panels include, without limitation: CA-125, cytokeratin-19, FasL, M-CSF; cytokeratin-19, CEA, Fas, EGFR, kallikrein-8; CEA, Fas, M-CSF, EGFR, CA-125; cytokeratin 19, kallikrein 8, CEA, CA 125, M-CSF; kallikrein-8, EGFR, CA-125; cytokeratin-19, CEA, CA-125, M-CSF, EGFR; cytokeratin-19, kallikrein-8, CA-125, M-CSF, FasL; cytokeratin-19, kallikrein-8, CEA, M-CSF; cytokeratin-19, kallikrein-8, CEA, CA-125; CA 125, cytokeratin 19, ErbB2; EGF, G-CSF, IL-6, IL-8, VEGF and MCP-1; anti-CA 15-3, anti-IL-8, anti-survivin, anti-p53 and anti c-myc; and anti-CA 15-3, anti-IL-8, anti-survivin, anti-p53, anti c-myc, anti-CEA, anti-IL-6, anti-EGF; and anti-bHCG.

The methods may further comprise comparing the levels of the two or more markers in the patient's blood with levels of the same markers in one or more a control samples by applying a statistical method such as: linear regression analysis, classification tree analysis and heuristic naïve Bayes analysis. The statistical method may be, and typically is performed by a computer process, such as by commercially available statistical analysis software. In one embodiment, the statistical method is a classification tree analysis, for example CART (C&RT, Classification and Regression Tree).

An array also is provided comprising binding reagent types specific to any two or more of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, CEA, kallikrein-8, M-CSF, EGFR and Her2/neu, wherein each binding reagent type is attached independently to one or more discrete locations on one or more surfaces of one or more substrates. The substrates may be beads comprising an identifiable marker, wherein each binding reagent type is attached to a bead comprising a different identifiable marker than beads to which a different binding reagent is attached. The identifiable marker may comprise a fluorescent compound or a quantum dot.

In another embodiment, a method is provided for determining the presence of ovarian cancer in a patient, comprising determining levels of at least one of anti-Her2/neu, anti-IL-8, anti-osteopontin, anti-VEGF and anti-PDGF in a sample of the patient's blood, where the presence of one or more of the following conditions indicates the presence of ovarian cancer in the patient: anti-Her2/neu_{HI}, anti-IL-8_{HI}, anti-osteopontin_{HI}, anti-VEGF_{HI}, anti-Akt1 and anti-PDGF_{HI}.

In a further embodiment, a method of predicting onset of clinical ovarian cancer is provided, comprising determining the change in concentration at two or more time points of two or more of anti-Her2/neu, anti-MUC-1, anti-c-myc, anti-p53, anti-CA-125, anti-CEA, anti-CA 72-4, anti-PDGFRa, IFNγ, IL-6, IL-10, TNFα, MIP-1α, MIP-1β, EGFR and Her2/neu in a patient's blood, wherein an increase in the concentration of anti-Her2/neu, anti-MUC-1, anti-c-myc, anti-p53, anti-CA-125, anti-CEA, anti-CA 72-4, anti-PDGFRα, IFNγ, IL-6 and IL-10 in the patient's blood between the two time points and a decrease in the concentration of TNFα, MIP-1α, MIP-1β, EGFR and Her2/neu in the patient's blood between the two time points are predictive of the onset of clinical ovarian cancer.

### BRIEF DESCRIPTION OF THE DRAWINGS

FIG. 1 are graphs showing serum markers in ovarian cancer patients and healthy controls.

FIG. 2 is a graph showing absorption of soluble EGF by ovarian carcinoma cells.

FIG. 3 provides graphs showing the distribution of serum levels of cytokines in the three study groups described in Example 3.

FIG. 4A provides a classification tree for discriminating early stage ovarian cancer from healthy controls.

FIG. 4B is a graph showing the ROC curve described in Example 4.

FIG. 5 provides graphs showing the distribution of serum levels of circulating antibodies in the three study groups in Example 6.

FIG. 6 provides graphs showing the distribution of serum levels of cancer markers in the three study groups of Example 6.

FIGS. 7A and 7B provides graphs showing the velocity of circulating serological markers in blood serum

### DETAILED DESCRIPTION

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Provided herein is a rapid, multifactorial assay for early and rapid identification of an ovarian malignancy. Identified below are blood cytokine, Immunoglobulin (Ig) and cancer antigen markers useful in the detection of ovarian cancer. Cytokine markers include: EGF, G-CSF, IL-6, IL-8, VEGF and MCP-1 that are abnormally expressed in the blood of patients with ovarian cancer. EGF and MCP-1 are under-expressed in patients with ovarian cancer, as compared to control individuals, while G-CSF, IL-6, IL-8 and VEGF are over-expressed in those patients. As such, there is a very high likelihood that a patient exhibiting two or more, and typically three or four of the following parameters: EGF_{LO}, G-CSF_{HI}, IL-6_{HI}, IL-8_{HI}, VEGF_{HI} or MCP-1_{LO} has ovarian cancer.

Also identified are certain Ig species that are present in abnormal levels in the blood of patients with ovarian cancer. These markers include antibodies against: IL-6, IL-8, CA-125, c-myc, p53, CEA, CA 15-3, MUC-1, survivin, bHCG, osteopontin, Her2/neu, Akt1, cytokeratin 19, and PDGF (Platelet Derived Growth Factor). As such, there is a very high likelihood that a patient exhibiting two or more, and typically three or four of the following conditions: anti-IL-6_{HI}, anti-IL-8_{HI}, anti-CA-125_{HI}, anti-c-myc_{HI}, anti-P53_{HI}, anti-CEA_{HI}, anti-CA 15-3_{HI}, anti-MUC-1_{HI}, anti-survivin_{HI}, anti-bHCG_{HI}, anti-osteopontin_{HI}, anti-Her2/neu_{HI}, anti-cytokeratin 19_{HI} and anti-PDGF_{HI} has ovarian cancer.

Also identified are certain cancer antigens that are present in abnormally high levels in the blood of patients with ovarian cancer. These markers include CA-125, FasL, CEA and cytokeratin 19. Other cancer antigens are present in abnormally low levels in ovarian cancer patients, including Her2/neu, M-CSF, kallikrein 8 and EGFR. As such, there is a very high likelihood that a patient exhibiting two or more, and typically three or four of the following conditions: CA-125_{HI}, cytokeratin 19_{HI}, EGFR_{LO}, Her2/neu_{LO}, CEA_{HI}, FasL_{HI}, kallikrein-8_{LO} and M-CSF_{LO} has ovarian cancer.

Panels of blood markers derived from each of the three groups described above also are useful in identifying whether a patient has ovarian cancer. Panels selected from two or more, typically three or four, of EGF, G-CSF, IL-6, IL-8, CA-125, VEGF, MCP-1, anti-IL6, anti-IL8, anti CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu also are useful in discriminating normal/benign patients from ovarian cancer patients. A number of markers are first described herein fro their usefulness in discriminating normal/benign patients from ovarian cancer patients. These novel ovarian cancer markers include: anti-Her2/neu, anti-IL-8, anti-VEGF, anti-osteopontin, anti-PDGF-M (Platelet Derived Growth Factor M homodimer) and anti-Akt1.

The parameters EGF_{LO}, G-CSF_{HI}, IL-6_{HI}, IL-8_{HI}, CA-125_{HI}, VEGF_{HI}, MCP-1_{LO}, anti-c-myc_{HI}, anti-p53_{HI}, anti-CEA_{HI}, anti-CA 15-3_{HI}, anti-MUC-1_{HI}, anti-survivin_{HI}, anti-bHCG_{HI}, anti-osteopontin_{HI}, anti-PDGF_{HI}, cytokeratin 19_{HI}, EGFR_{LO}, Her2/neU_{LO}, CEA_{HI}, FasL_{HI}, kallikrein-8_{LO} and M-CSF_{LO} are determined statistically by comparing normal or control blood (serum or plasma) levels of these markers with blood levels in patients with ovarian cancer. The statistical data presented below identifies certain values defining certain _{LO} or _{HI} parameters for the above-described markers in patients. As a non-limiting example of estimates of _{LO} and _{HI} values, in reference to the data of Example 1, EGF_{LO} means less than about 224 pg/mL EGF, G-CSF_{HI} means greater than about 22 pg/mL G-CSF, IL-6_{HI} means greater than about 8.8 pg/mL IL-6, IL-8_{HI} means greater than about 10.2 pg/mL IL-8, CA-125_{HI}, means greater than about 10 pg/mL CA-125, VEGF_{HI} means greater than about 91 pg/mL VEGF or MCP-1_{LO} means less than about 342 pg/mL MCP-1. Identification of _{LO} and _{HI} values for other markers identified herein, including, without limitation, EGF_{LO}, G-CSF_{HI}, IL-6_{HI}, IL-8_{HI}, CA-125_{HI}, VEGF_{HI}, MCP-1_{LO}, anti-c-myc_{HI}, anti-p53_{HI}, anti-CEA_{HI}, anti-CA 15-3_{HI}, anti-MUC-1_{HI}, anti-survivin_{HI}, anti-bHCG_{HI}, anti-osteopontin_{HI}, anti-PDGF_{HI}, cytokeratin 19_{HI}, EGFR_{LO} and Her2/neu_{LO}, can be determined by reference to the graphs provided herein, the data presented herein and/or by use of statistical methods as described herein, all of which are within the abilities of a person of ordinary skill in the field of biostatistics based on the data presented herein.

It is understood that these _{LO} and _{HI} values are approximate and are derived statistically. By using other statistical methods to detect the relative levels of each factor and to define the critical values for _{HI} and _{LO}, values slightly above or below, typically within one standard deviation of those approximate values might be considered as statistically significant values for distinguishing the _{LO} or _{HI} state from normal. For this reason, the word “about” is used in connection with the stated values. “Statistical classification methods” are used to identify markers capable of discriminating normal patients and patients with benign growths with ovarian cancer patients, and are used to determine critical blood values for each marker for discriminating such patients. Three particular statistical methods were used to identify discriminating markers and panels thereof. These statistical methods include: 1) linear regression, as identified in Example 1, below; 2) classification tree methods (CART, as used in the examples below, along with CHAID and QUEST are classification tree programs), as identified in Example 4, below; and 3) statistical machine learning to optimize the unbiased performance of algorithms for predicting the masked class labels as described in Example 7, below. Each of these statistical methods are well-know to those of ordinary skill in the field of biostatistics and can be performed as a process in a computer. A large number of software products are available commercially to implement statistical methods, such as, without limitation, S-PLUS®, commercially available from Insightful Corporation of Seattle, Wash.

By identifying markers present in ovarian cancer patients and statistical methods useful in identifying which markers and groups of markers are useful in identifying ovarian cancer patients, a person of ordinary skill in the art, based on the disclosure herein, can identify panels that provide superior selectivity and sensitivity. Examples of panels providing excellent discriminatory capability include, without limitation: CA-125, cytokeratin-19, Fas, M-CSF; cytokeratin-19, CEA, Fas, EGFR, kallikrein-8; CEA, Fas, M-CSF, EGFR, CA-125; cytokeratin 19, kallikrein 8, CEA, CA 125, M-CSF; kallikrein-8, EGFR, CA-125; cytokeratin-19, CEA, CA-125, M-CSF, EGFR; cytokeratin-19, kallikrein-8, CA-125, M-CSF, Fas; cytokeratin-19, kallikrein-8, CEA, M-CSF; cytokeratin-19, kallikrein-8, CEA, CA-125; CA 125, cytokeratin 19, ErbB2; EGF, G-CSF, IL-6, IL-8, VEGF and MCP-1; anti-CA 15-3, anti-IL-8, anti-survivin, anti-p53 and anti c-myc; and anti-CA 15-3, anti-IL-8, anti-survivin, anti-p53, anti c-myc, anti-CEA, anti-IL-6, anti-EGF; and anti-bHCG. It will be recognized by those of ordinary skill in the field of biostatistics, that the number of markers in any given panel may be different depending on the combination of markers. With optimum sensitivity as specificity being the goal, one panel may include two markers, while another may include eight, both yielding similar results.

The term “binding reagent” and like terms, refers to any compound, composition or molecule capable of specifically or substantially specifically (that is with limited cross-reactivity) binding another compound or molecule, which, in the case of immune-recognition is an epitope. A “binding reagent type” is a binding reagent or population thereof having a single specificity. The binding reagents typically are antibodies, preferably monoclonal antibodies, or derivatives or analogs thereof, but also include, without limitation: Fv fragments; single chain Fv (scFv) fragments; Fab′ fragments; F(ab′)₂ fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments. “Binding reagents” also include aptamers, as are described in the art.

Methods of making antigen-specific binding reagents, including antibodies and their derivatives and analogs and aptamers, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163, 5,475096, 5,840867 and 6,544,776.

The ELISA and Luminex LabMAP immunoassays described below are examples of sandwich assays. The term “sandwich assay” refers to an immunoassay where the antigen is sandwiched between two binding reagents, which are typically antibodies. The first binding reagent/antibody being attached to a surface and the second binding reagent/antibody comprising a detectable group. Examples of detectable groups include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

In the bead-type immunoassays described in the examples below, the Luminex LabMAP system is utilized. The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are far superior for quantitation purposes because the bead technology does not require pre-processing or titering of the plasma or serum sample, with its inherent difficulties in reproducibility, cost and technician time. For this reason, although other immunoassays, such as, without limitation, ELISA, RIA and antibody microarray technologies, are capable of use in the context of the present invention, but they are not preferred. As used herein, “immunoassays” refer to immune assays, typically, but not exclusively sandwich assays, capable of detecting and quantifying a desired blood marker, namely one of EGF, G-CSF, IL-6, IL-8, CA-125, VEGF, MCP-1, anti-IL6, anti-IL8, anti CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu.

Data generated from an assay to determine blood levels of two, three or four or more of the markers EGF, G-CSF, IL-6, IL-8, CA-125, VEGF, MCP-1, anti-IL6, anti-IL8, anti CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu can be used to determine the likelihood of an ovarian cancer in the patient. As shown herein, if any two or more, typically three or four of the following conditions are met in a patient's blood, EGF_{LO}, G-CSF_{HI}, IL-6_{HI}, IL-8_{HI}, VEGF_{HI}, MCP-1_{LO}, anti-IL-6_{HI}, anti-IL-8_{HI}, anti-CA-125_{HI}, anti-c-Myc_{HI}, anti-p53_{HI}, anti-CEA_{HI}, anti-CA 15-3_{HI}, anti-MUC-1_{HI}, anti-survivin_{HI}, anti-bHCG_{HI}, anti-osteopontin_{HI}, anti-Her2/neu_{HI}, anti-Akt1_{HI}, anti-cytokeratin 19_{HI} and anti-PDGF_{HI}, CA-125_{HI}, cytokeratin 19_{HI}, EGFR_{LO}, Her2/neu_{LO}, CEA_{HI}, FasL_{HI}, kallikrein-8_{LO}, ErbB2_{LO} and M-CSF_{LO}, there is a very high likelihood that the patient has ovarian cancer. In one embodiment, if any three or more, preferably three or four of the following conditions are met in a patient's blood, EGF_{LO}, G-CSF_{HI}, IL-6_{HI}, IL-8_{HI}, VEGF_{HI}, MCP-1_{LO}, anti-IL-6_{HI}, anti-IL-8_{HI}, anti-CA-125_{HI}, anti-c-myc_{HI}, anti-p⁵³_{HI}, anti-CEA_{HI}, anti-CA 15-3_{HI}, anti-MUC-1_{HI}, anti-survivin_{HI}, anti-bHCG_{HI}, anti-osteopontin_{HI}, anti-Her2/neu_{HI}, anti-Akt1_{HI}, anti-cytokeratin 19_{HI}, and anti-PDGF_{HI}, CA-125_{HI}, cytokeratin 19_{HI}, EGFR_{LO}, Her2/neu_{LO}, CEA_{HI}, FasL_{HI}, kallikrein-8_{LO}, ErbB2_{LO} and M-CSF_{LO}, there also is a very high likelihood that the patient has ovarian cancer.

In the context of the present diclosure, “blood” includes any blood fraction, for example serum, that can be analyzed according to the methods described herein. Serum is a standard blood fraction that can be tested, and is tested in the Examples below. By measuring blood levels of a particular marker, it is meant that any appropriate blood fraction can be tested to determine blood levels and that data can be reported as a value present in that fraction. As a non-limiting example, the blood levels of a marker can be presented as 50 pg/mL serum.

As described above, methods for diagnosing ovarian cancer by determining levels of specific identified blood markers are provided. Also provided are methods of detecting preclinical ovarian cancer comprising determining the presence and/or velocity of specific identified markers in a patient's blood. By velocity it is meant the changes in the concentration of the marker in a patient's blood over time. Example 7 provides longitudinal data showing the value of determining the velocity of specific markers in a patient's blood in predicting onset of clinical ovarian cancer. Markers with demonstrable velocity indicative of preclinical ovarian cancer include: anti-Her2/neu, anti-MUC-1, anti-c-myc, anti-p53, anti-CA-125, anti-CEA, anti-CA 72-4, anti-PDGFRα, IFNγ, IL-6 and IL-10, which increase in concentration beginning at 30-40 months prior to clinical onset of ovarian cancer; and TNFα, MIP-1α, MIP-1β, EGFR and Her2/neu, which decrease in concentration beginning at 30-40 months prior to clinical onset of ovarian cancer.

### EXAMPLE 1

Patient Population. Serum samples from 55 patients diagnosed with early (I-II) stages ovarian cancer, 55 patients with benign pelvic masses, and 55 healthy age-matched controls were tested. Serum samples from patients with early stages (I-II) ovarian cancer and women with benign pelvic disease, were provided by the Gynecologic Oncology Group (GOG) (Cleveland, Ohio). Consent and blood specimens from all participants were obtained under IRB Protocol. Charts were reviewed by clinical oncologist to verify gynecologic diagnoses and ovarian cancer staging. Pathology slides for ovarian cancer cases were reviewed by a pathologist to verify histology and grade. All major types of epithelial ovarian cancer and benign pelvic conditions were represented. Table A summarizes patient data. Control serum samples from healthy, age-matched women were received from the Allegheny County Case-Control Network under the IRB Protocol.

TABLE A
Patient characteristics

| Patient Group | Age | Histologic Types |
|---|---|---|
| Control | Range 23-76 | |
| N = 55 | Median 46 | |
| Early Stage OvCa | Range 14-88 | Papillary serous carcinoma (n = 18) |
| N = 55 | Median 46 | Adenocarcinoma, Endometrioid (n = 8) |
| | | Carcinoma, Endometroid (n = 4) |
| | | Adenocarcinoma, Mucinous (n = 5) |
| | | Carcinoma, Mucinous (n = 3) |
| | | Adenocarcinoma, Poorly Differentiated |
| | | (n = 3) |
| | | Carcinoma, Poorly Differentiated |
| | | (n = 3) |
| | | Adenocarcinoma, Serous (n = 8) |
| | | Carcinoma, Clear Cell (n = 3) |
| Benign | 15-87 | Adenofibroma, Serous (n = 1) |
| N = 55 | 55.1 ± 15.3 | Brenner Tumor (n = 1) |
| | 38.5 | Crystadenofibroma, Serous (n = 2) |
| | | Cyst, Paratubal (n = 2) |
| | | Cyst, Serous (n = 1) |
| | | Cyst, Simple (n = 3) |
| | | Cystadenofibroma, Serous (n = 3) |
| | | Cystadenoma, Mucinous (n = 10) |
| | | Cystadenoma, Serous (n = 11) |
| | | Endometriosis (n = 1) |
| | | Fibrosis (n = 1) |
| | | Ovary benign (n = 3) |
| | | Mucinous benign (n = 2) |

Collection and storage of blood specimens: Ten mL of peripheral blood was drawn from subjects using standardized phlebotomy procedures. Blood samples were collected without anticoagulant into two 5 mL red top vacutainers, sera were separated by centrifugation, and all specimens were immediately frozen and stored in the dedicated −80° C. freezer. All blood samples were logged on the study computer to track information such as storage date, freeze/thaw cycles and distribution.

Multiplex Analysis was performed using multiplexed kits purchased from BioSource International (Camarillo, Calif.) according to manufacturer's protocol. The minimum cytokine detection level for these kits is <5 pg/mL. The following 29 cytokines, angiogenic, death and growth factors were analyzed in a multiplex format: IL-10, IL-2, IL-4, IL-5, IL-6, IL-8, IL-10, IL-12p40, IL-13, IL-15, IL-17, IL-18, TNFα (Tumor Necrosis Factor α), IFNγ (Interferon γ), GM-CSF (Granulocyte Macrophage Colony Stimulating Factor), EGF, VEGF, G-CSF, bFGF (basic Fibroblast Growth Factor), HGF (Hepatocyte Growth Factor), RANTES (Regulated on Activation, Normal T Expressed and Secreted, also known as CCL5 or MCP2), MIP-1α (macrophage inflammatory protein-1 alpha), MIP-1β (macrophage inflammatory protein-1 beta), MCP-1, EGFR (epidermal growth factor receptor), TGFβ (Transforming Growth Factor beta), FasL (Fas Ligand), survivin and CA-125.

The assays were performed in 96-well microplate format. A filter-bottom 96-well microplate (Millipore) was blocked for 10 min with PBS/BSA. To generate a standard curve, serial dilutions of appropriate standards provided by manufacturers were prepared in serum diluent. Standards and patients sera were pipetted at 50 μl/well in duplicate and mixed with 50 μl of bead mixture. Microplate was incubated for 1 h at room temperature on microtiter shaker. Wells were then washed three times with washing buffer using a vacuum manifold. PE-conjugated secondary antibody were added to the appropriate wells and incubated for 45 min in the dark with the constant shaking. Wells were washed twice, assay buffer was added to each well and samples were analyzed using the Bio-Plex suspension array system, which includes a fluorescent reader and Bio-Plex Manager analytical software (Bio-Rad Laboratories, Hercules, Calif.). Data analysis was done with using five-parametric-curve fitting.

Development of Luminex assay. VEGF, G-CSF IL-6, IL-8, IL-12p40, EGF, MCP-1, and CA-125 reagents for multiplex system were developed using antibody pairs purchased from R&D Systems (Minneapolis, Minn.) for all analytes except CA-125, and Fitzgerald Industries International (Concord, Mass.) for CA-125 (Table B). Capture antibodies were monoclonal and detection antibodies were polyclonal. Capture Abs were covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath (Sonicor Instrument Corporation, Copiaque, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μl. This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/mL, were prepared in phosphate buffer, and 10 μl of each solution was sequentially added to stabilize the reaction and activate the microspheres. This suspension was incubated for 10 min at room temperature and then resuspended in 250 μl of PBS containing 50 μg of antibody. The mixture was incubated overnight in the dark with continuous shaking. Microspheres were then incubated with 250 μl of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4° C. Coupling efficiency of monoclonal antibodies was tested by staining 2,000 microspheres with PE-conjugated goat anti-mouse IgG (BD Biosciences, San Diego, Calif.). Detection Abs were biotinylated using EZ-Link Sulfo-NHS-Biotinylation Kit (Pierce, Rockford, Ill.) according to manufacturer's protocol. The extent of biotin incorporation was determined using HABA assay and was 20 moles of biotin per mole of protein. The assays were further optimized for concentration of detection Ab and for incubation times. Sensitivity of the newly developed assays were determined using serially diluted purified proteins. Intra-assay variability, expressed as a coefficient of variation, was calculated based on the average for 10 patient samples and measured twice at two different time points. The intra-assay variability within the replicates presented as an average coefficient of variation was 8.5% (data not shown). Inter-assay variability was evaluated by testing quadruplicates of each standard and 10 samples and was between 10 and 22%, with an average of 16.5% (data not shown). Newly developed kits were multiplexed together and the absence of cross-reactivity was confirmed according to Luminex protocol.

TABLE B
Commercial Sources of Antibodies

| | | Matched Antibody Pair |
|---|---|---|
| Cytokine | Commercial Source | Identifier/Catalog No. |
| EGF | R&D Systems | MAB636 |
| | (Minneapolis, MN) | BAF236 |
| G-CSF | R&D Systems | DY214 |
| IL-6 | R&D Systems | DY206 |
| IL-8 | R&D Systems | DY208 |
| IL-12p40 | R&D Systems | DY1240 |
| MCP-1 | R&D Systems | DY279 |
| VEGF | R&D Systems | DY293 |
| CA-125 | Fitzgerald Industries | M002201 |
| | International, Inc. | M002203 |
| | (Concord, MA) | |

Additionally, CA-125 reagent for multiplex system was developed using antibody pair purchased from Fitzgerald Industries International (Concord, Mass.). Capture antibody was monoclonal and detection antibody was sheep polyclonal. Capture Ab was biotinylated using EZ-Link Sulfo-NHS-Biotinylation Kit (Pierce, Rockford, Ill.) according to the manufacturer's protocol. The extent of biotin incorporation was determined using HABA assay and was 20 moles of biotin per mole of protein. Capture Ab was covalently coupled to carboxylated polystyrene microspheres number 74 purchased from Luminex Corporation (Austin, Tex.). Covalent coupling of the capture antibodies to the microspheres was performed byfollowing the procedures recommended by Luminex. In short, the microspheres' stock solutions were dispersed in a sonification bath (Sonicor Instrument Corporation, Copiaque, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μl. This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/m L, were prepared in phosphate buffer, and 10 μl of each solution was sequentially added to stabilize the reaction and activate the microspheres. This suspension was incubated for 10 min at room temperature and then resuspended in 250 μl of PBS containing 50 μg of antibody. The mixture was incubated overnight in the dark with continuous shaking. Microspheres were then incubated with 250 μl of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4° C. Coupling efficiency of monoclonal antibodies was tested by staining 2,000 microspheres with PE-conjugated goat anti-mouse IgG (BD Biosciences, San Diego, Calif.). The assay was further optimized for concentration of detection Ab and for incubation times. Sensitivity of the newly developed assay as determined in a Luminex assay using serially diluted purified CA-125, was 20 IU. Intra-assay variability, expressed as a coefficient of variation, was calculated based on the average for 10 patient samples and measured twice at two different time points. The intra-assay variability within the replicates presented as an average coefficient of variation was 8.5% (data not shown). Interassay variability was evaluated by testing quadruplicates of each standard and 10 samples. The variabilities of these samples were between 10 and 22%, with an average of 16.5% (data not shown). Next, the anti-CA-125 microsphereswere combined with the existing multiplex kit.

Statistical Analysis of Data. All statistical analyses were conducted using S-Plus statistical software (Seattle, Wash.: Math Soft, Inc., 1999). The data were first randomly split into a training and test set; described in Table C. Logistic regression (Hosmer, D W, S Lemeshow, Applied Logistic Regression. New York, N.Y.: John Wiley & Sons, 1989) was then used to calculate the optimal weighting of each marker and the subsequent predicted probability of being a case. All predicted probabilities ≧0.5 were categorized as a predicted case; predicted probabilities <0.5 were categorized as a predicted control. After fitting a logistic model to the training set, classification of disease status was then calculated for the test set.

TABLE C
Training and test sets for comparison
of controls versus early stage disease

| | | | #Early Stage |
|---|---|---|---|
| Data Set | Total N | #Controls | Cancers |
| All Data | 87 | 41 | 46 |
| Training Data | 43 | 20 | 23 |
| Test Data | 44 | 21 | 23 |

Cytokines. Recombinant VEGF, EGF and MCP-1 were purchased from commercial sources. Recombinant IL-6, IL-8 and IL-12 were obtained from PeproTech, Inc (Rocky Hill, N.J.). Polyclonal neutralizing anti-EGF Ab (Ab 528) was obtained from R&D Systems, Inc. (Minneapolis, Minn.).

Results

Serum concentrations of cytokines and angiogenic factors by LabMap technology. Circulating concentrations of 26 different serum markers (Table D) were evaluated in a multiplexed assay using LabMap technology in blood of patients from three clinical groups, control healthy volunteers, women with benign pelvic masses, and women with early stages ovarian cancer.

TABLE D
Serum markers

| | | | Angiogenic | Growth | Death | Cancer |
|---|---|---|---|---|---|---|
| Groups | Cytokines | Chemokines | factors | factors | factors | antigens |
| Markers | IL-1β, IL-2, | MCP-1, | VEGF, | EGF, | FasL, | CA-125 |
| | IL-4, IL-5, | MIP-1α, | bFGF, | EGFR, | Survivin | |
| | IL-6, IL-8, | MIP-1β | IL-6, | HGF, | | |
| | IL-10, IL-12p40, | | IL-8 | TGFβ | | |
| | IL-13, IL-15, | | | | | |
| | IL-17, IL-18, | | | | | |
| | TNFα, IFNγ, | | | | | |
| | G-CSF, GM-CSF, | | | | | |
| | RANTES | | | | | |

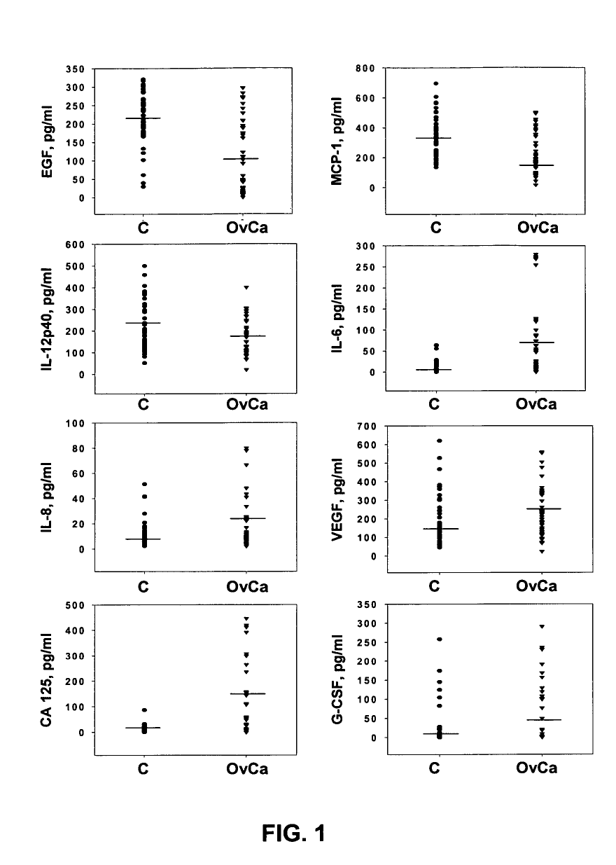

Serum levels of IL-2, IL-4, IL-5, IL-10, IL-13, IL-15, IL-17, IL-18, TNFα, IFNγ, RANTES, GM-CSF, bFGF and survivin were undetectable in either control or patient groups. IL-1β, MIP-1α, MIP-1β, HGF, TGFβ, EGFR and FasL demonstrated measurable serum concentrations, which did not differ between the control and patient groups (data not shown). Serum concentrations of IL-6, IL-8, G-CSF, VEGF, and CA-125 were significantly (P<0.01) higher in ovarian cancer patients as compared to controls. Surprisingly, women with ovarian cancer demonstrated significantly lower blood levels of EGF, IL-12p40 and MCP-1 (p<0.001). The results are presented for EGF, G-CSF, IL-6, IL-8, CA-125, VEGF, MCP-1 and IL-12p40 in Table E and FIG. 1.

TABLE E
Levels of serum markers

| | | | Ovarian | | |
|---|---|---|---|---|---|
| Analyte | | Controls | Cancer | Benign | Phase III-IV |
| EGF | Range | 29.8-402.6 | 0-396.9 | 0-276.4 | 7.4-333.0 |
| | Mean ± SE | 223.8 ± 11.88 | 110.7 ± 15.58 | 98.6 ± 12.35 | 113.0 ± 14.94 |
| | Median | 238 | 74.9 | 94.9 | 93.2 |
| IL-6 | Range | 0-64.1 | 0-280.2 | 0-275.3 | 0-454.2 |
| | Mean ± SE | 8.8 ± 2.50 | 64.2 ± 12.72 | 28.0 ± 9.3 | 65.3 ± 12.52 |
| | Median | 0 | 23.8 | 7.6 | 38.0 |
| G-CSF | Range | 0-257.6 | 0-290.8 | 0-339.1 | 0-732.8 |
| | Mean ± SE | 21.8 ± 8.44 | 49.2 ± 12.04 | 77.4 ± 14.04 | 71.7 ± 20.61 |
| | Median | 0 | 0 | 0 | 0 |
| IL-8 | Range | 2.3-51.4 | 2.0-180.6 | 3.0-127.8 | 4.1-52.6 |
| | Mean ± SE | 10.2 ± 1.68 | 24.0 ± 5.98 | 12.4 ± 3.11 | 14.4 ± 1.68 |
| | Median | 6 | 9.6 | 7.6 | 11.0 |
| VEGF | Range | 18-306 | 28-552 | 48-662 | 22-954 |
| | Mean ± SE | 90.7 ± 10.52 | 153.5 ± 19.95 | 258.8 ± 26.04 | 263.8 ± 38.29 |
| | Median | 67 | 106 | 218 | 170 |
| CA-125 | Range | 0-87 | 0-1412 | 0-372 | 0-2512 |
| | Mean ± SE | 10.4 ± 2.28 | 153.7 ± 44.04 | 51.8 ± 13.23 | 269.1 ± 895.60 |
| | Median | 6.0 | 51.0 | 16.0 | 55.0 |
| IL-12p40 | Range | 52.3-500.0 | 20.0-400.0 | 84.0-360.4 | 20.8-327.4 |
| | Mean ± SE | 210.7 ± 17.22 | 170.0 ± 13.38 | 169.2 ± 10.69 | 157.3 ± 10.49 |
| | Median | 162.4 | 149.8 | 151.2 | 149.6 |
| MCP-1 | Range | 135.5-695.7 | 17.1-502.3 | 44.9-434.6 | 38.3-534.0 |
| | Mean ± SE | 341.8 ± 21.34 | 210.3 ± 20.54 | 196.3 ± 16.06 | 228.5 ± 21.29 |
| | Median | 326.8 | 172.9 | 178.2 | 201.2 |

Statistical Analysis. To evaluate prognostic ability of these cytokines, the data were first randomly split into a training set and a test set of approximately equal size. For each comparison of interest, (i.e. controls versus early stage cancer, controls versus benign, and benign versus early stage cancer), a logistic regression model (Hosmer et al., 1989.) was first fit to the training data; predicted probabilities (of being a case) and classification results were then obtained using the independent test set. The random selection of test and training data was repeated 1,000 times for each model to obtain valid estimates for the variability of classification rates. Results were described in terms of the mean (across all 1,000 random partitions of the training and test sets) percent correctly classified (PCC), sensitivity (SEN), and specificity (SPC). The 95% confidence intervals (95% Cl) for PCC, SEN, and SPC were also displayed as the 2.5 and 97.5 percentiles of the distribution. All statistical analyses were conducted using S-Plus statistical software (Seattle, Wash.: Math Soft, Inc., 1999).

In general, the logistic model with k variables (i.e. cytokines) is represented by the following equation where ŷ is the predicted case status and x₁ to xₖ are the expression levels for the cytokines of interest.

$$ln ( \frac{\hat{y}}{1 - \hat{y}} ) = {β}_{0} + {β}_{1} {x}_{1} + {β}_{2} {x}_{2} + … + {β}_{k} {x}_{k}$$

The log function (i.e. the left-hand side of the equation) transforms the dichotomous outcome (i.e. case or control) into a quantity that is linear in the log scale.

Using coefficients from the logistic model, as fit to the training data, the predicted probability of being a case was then calculated for each subject in the test set. If the predicted probability of being a case was higher than the observed proportion of cases in the training set (usually just over 0.5), the subject was then classified as a predicted case. If the predicted probability of being a case was lower than the observed proportion of cases in the training set, the subject was then classified as a predicted control. Fitting the logistic model on one data set, and then predicting the outcome for an independent (i.e. randomly selected) test set allows for unbiased estimation of classification accuracy, sensitivity, and specificity.

For a given comparison (e.g. controls versus early stage cancer), the logistic model was initially fit to each individual cytokine. The cytokine leading to the highest classification rate (i.e. percentage correctly classified) was then separately entered into a series of 2-variable models with each of the remaining cytokines. For instance, if EGF produced the best classification, each of the remaining cytokines would then be entered into a 2-variable model with EGF. The 2-variable model producing the highest percentage correctly classified was then separately combined with each of the remaining cytokines to form a series of 3-variable models. A similar step-up, or forward selection procedure was continued as long as similar or better classification accuracy was achieved with the larger model. The model producing the highest classification rate was denoted as the optimal model.

Comparison of controls versus early stage ovarian cancer. Table F illustrates classification results when using each individual cytokine to identify early stage ovarian cancer from controls. Results show that none of these cytokines individually led to extremely accurate prediction of early stage cancer. Only EGF correctly classified over 75% of the test set subjects. Only two other cytokines (MCP and IL-6) led to over 60% correctly classified. However, the 95% confidence intervals indicate that three of the nine cytokines (EGF, MCP, and IL-6) individually showed significantly better-than-chance classification, i.e. the lower 95% confidence limit for the PCC was above 50.0%.

TABLE F
Classification Using a Single Marker
to Predict Early Stage from Controls

| | % Correctly | | |
|---|---|---|---|
| | Classified | Sensitivity | Specificity |
| Cytokine | [95% Cl] | [95% Cl] | [95% Cl] |
| EGF | 73.2 [65.9, 79.5] | 65.9 [50.0, 81.8] | 80.4 [63.6, 95.5] |
| VEGF | 54.4 [39.5, 68.2] | 50.6 [22.7, 95.5] | 58.2 [13.6, 81.0] |
| MCP-1 | 68.6 [61.4, 77.3] | 71.1 [59.1, 81.8] | 66.0 [50.0, 77.3] |
| IL-6 | 68.4 [61.4, 75.0] | 51.5 [36.4, 68.2] | 85.2 [68.2, 95.5] |
| IL-8 | 56.3 [47.7, 63.6] | 32.6 [18.2, 50.0] | 80.0 [63.6, 95.5] |
| IL-12 | 52.6 [43.2, 61.4] | 61.9 [40.9, 81.8] | 43.3 [27.3, 59.1] |
| G-CSF | 57.9 [50.0, 65.9] | 32.3 [22.7, 45.5] | 83.5 [68.2, 95.5] |
| CA-125 | 75.6 [67.4, 83.7] | 63.4 [50.0, 77.3] | 88.4 [71.4, 100.0] |

Since EGF was the most predictive of early stage cancer, it was entered first into the model selection process. The additional models were formulated by continuing the forward selection process as described above. Table G shows the resulting multiple regression models. Results show that the model with four cytokines led to the best classification rate, and was therefore selected as the optimal model. One model with EGF, IL-6, IL-8 and VEGF led to over 90% accuracy in terms of correct classification (90%), sensitivity (90%), and specificity (91%). Additional models, with six or more cytokines led to decreasing classification rates (not shown here).

TABLE G
Classification Using Multiple Markers
to Predict Early Stage from Controls

| | % Correctly | | |
|---|---|---|---|
| | Classified | Sensitivity | Specificity |
| Optimal Models | [95% Cl] | [95% Cl] | [95% Cl] |
| CA-125 + MCP-1 | 84.4 | 80.3 | 88.6 |
| | [76.7, | [63.6, | [72.7, |
| | 92.9] | 90.9] | 100.0] |
| CA-125 + MCP-1 + IL-6 | 86.4 | 84.8 | 88.1 |
| | [77.3, | [68.2, | [71.4, |
| | 93.2] | 95.5] | 100.0] |
| CA-125 + MCP-1 + IL-6 + EGF | 87.5 | 88.4 | 86.5 |
| | [79.1, | [77.3, | [71.4, |
| | 93.2] | 100.0] | 100.0] |
| CA-125 + MCP-1 + IL-6 + EGF + | 88.7 | 89.2 | 88.2 |
| IL-8 | [79.1, | [72.7, | [72.7, |
| | 95.3] | 100.0] | 100.0] |

Cytokine levels in supernatants of cultured ovarian carcinoma cells. To substantiate the in vivo data, the levels of IL-6, IL-8, G-CSF, VEGF, EGF, IL-12p40 and MCP-1 in cell culture media of two ovarian carcinoma cell lines, OVCAR3 and SKOV3 were evaluated. Luminex bead analysis revealed measurable levels of VEGF, IL-6, IL-8, and G-CSF in conditioned culture media of both cell lines, indicating the secretion of the above cytokines by ovarian carcinoma cells. In contrast, no measurable EGF, IL-12p40 or MCP-1 could be identified in conditioned culture medium (data not shown).

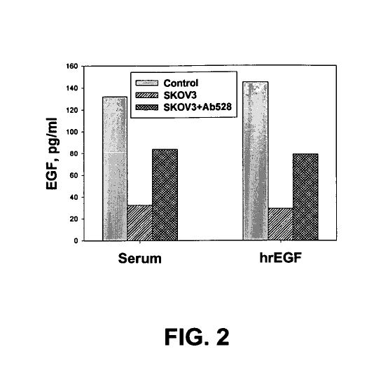

These in vivo results demonstrate lower circulating concentrations of EGF, MCP-1 and IL-12. It was hypothesized that the decreased levels of these cytokines are due to consumption by tumor. To ascertain this hypothesis, 10⁸ of each OVCAR3 and SKOV3 ovarian carcinoma cells were incubated with 100 μl of blood serum of women containing measurable concentrations of all three cytokines, for 1 hr at RT. Complete depletion of these three cytokines from sera after 1 hr incubation was observed. Furthermore, both ovarian carcinoma cells lines consumed EGF, MCP-1 and IL-12 from PBS, or from spiked sera. When specific binding of EGF was inhibited by addition of specific neutralizing Ab, no EGF depletion from sera could be observed (FIG. 2). No depletion of recombinant IL-6, IL-8 or VEGF from PBS by ovarian carcinoma cells could be observed (data not shown).

The Luminex LabMap detection assay utilizing differentially dyed fluorescent beads has a clear advantage above the conventional ELISA, that is, the ability to detect large numbers of analytes simultaneously at a sensitivity, accuracy, and reproducibility comparable to the ELISA (Veikkola et al., 2000). Using the LabMAP technique for screening of blood sera of women with early stage ovarian cancer in comparison with normal controls, eight circulating proteins were identified with ovarian cancer specificity, EGF, MCP-1, IL-12p40, G-CSF, CA-125, VEGF, IL-6 and IL-8. Circulating levels of all these proteins were close to those measured by ELISA or RIA and reported in published observations.

Two distinct patterns of cytokine levels were observed in ovarian cancer as compared to control. VEGF, IL-6, IL-8 and CA-125 were elevated in blood of ovarian cancer patients. In addition, higher levels of circulating G-CSF in patients with ovarian cancer was observed for the first time. Increased levels of cytokines in blood of cancer patients may be due to secretion by tumor or by non-tumor cells, that is, immune or endothelial cells in response to tumor. In agreement with published observations (Santin et al., 1999), IL-6, G-CSF (Glezerman et al., Tumor necrosis factor-alpha and interleukin-6 are differently expressed by fresh human cancerous ovarian tissue and primary cell lines. Eur Cytokine Netw. 1998 June;9(2):171-9 and Ziltener et al., Secretion of bioactive interleukin-1, interleukin-6, and colony-stimulating factors by human ovarian surface epithelium. Biol Reprod. 1993 September;49(3):635-41), and IL-8 (Xu, L. and I. J. Fidler, Interleukin 8: an autocrine growth factor for human ovarian cancer. Oncol Res, 2000. 12(2): p. 97-106), the in vitro secretion of VEGF was observed by ovarian carcinoma cells. However, these cytokines can also be produced by other cells, for example, VEGF can be produced and secreted by several normal cell types including smooth muscle, luteal and adrenal cortex cells; IL-6, IL-8 and MCP-1 (CCL2) can be can be produced by many cells, including macrophages, dendritic cells, endothelial cells, fibroblasts, and lymphoid cells. Tumor-secreted factors would be tumor-type specific, but theoretically would become measurable only upon tumor reaching certain size. An example of such tumor marker is CA-125, which is elevated in 85% of late stages epithelial ovarian cancers, but only in less than 50% of patients with stage I disease. On the other hand, cytokines induced in response to growing tumor in immune and other cells would show less tumor specificity but may become elevated during early stages of tumor development. Ideally, a diagnostic test should measure the combination of markers representing both groups.

A different pattern was demonstrated by EGF, MCP-1 and IL-12p40, which were lower in ovarian cancer as compared to control sera. Of eight studied antigens, EGF showed the strongest association with ovarian cancer. This is the first description of decreased EGF levels with strong association with disease in patients with ovarian cancer. Decreased circulating EGF levels were observed in patients with differentiated carcinoma of thyroids (Nedvidkova et al., Epidermal growth factor (EGF) in serum of patients with differentiated carcinoma of thyroids Neoplasma. 1992;39(1): 11-4), but not in patients with breast cancer or melanoma (our unpublished observation). Therefore, decreased circulating levels of EGF may be cancer-specific. Ovarian cancer cells express EGF receptor and EGF is autocrine growth factor for ovarian cells (Baron, A. T., et al., Serum sErbB1 and epidermal growth factor levels as tumor biomarkers in women with stage III or IV epithelial ovarian cancer. Cancer Epidemiol Biomarkers Prev, 1999. 8(2): p. 129-37 and Maihle, N. J., et al., EGF/ErbB receptor family in ovarian cancer. Cancer Treat Res, 2002. 107: p. 247-58). As our in vitro experiments indicate, lower circulating EGF levels in ovarian cancer patients might be due to the consumption of EGF by ovarian tumor cells. In addition, it was shown that soluble EGF receptor (sErbB1) could be found in the blood of late these patients (Baron et al., 1999 and Maihle et al., 2002). EGFR/EGF interaction might additionally increase clearance of EGF, resulting in the reduction of the blood level of EGF in ovarian cancer patients. It should be noted, that contrary to the above cited publications (Baron et al., 1999 and Maihle et al., 2002), ovarian cancer-specific differences in circulating concentration of ErbB1 by LabMap method was not observed. Similar to EGF, early stage ovarian cancer patients demonstrated lower levels of circulating MCP-1. Similar to the observations presented herein, lower circulating levels of MCP-1 in ovarian cancer as compared to control were noted by Penson et al. (Cytokines IL-1beta, IL-2, IL-6, IL-8, MCP-1, GM-CSF and TNFalpha in patients with epithelial ovarian cancer and their relationship to treatment with paclitaxel, Int J Gynecol Cancer 2000 January;10(1):33-41). However, in another study, higher circulating levels of MCP-1 in ovarian cancer patients as compared to controls were reported (Hefler et al., Monocyte chemoattractant protein-1 serum levels in ovarian cancer patients Br J Cancer. 1999 November;81(5):855-9).

Statistical analysis demonstrated that although correlation of each of the above markers with ovarian cancer was modest, a combined panel consisting of three or four of these markers showed very strong association with disease, and can therefore be used for early diagnosis of ovarian cancer. Several models provided comparable high sensitivity and specificity for early diagnosis of ovarian cancer. Therefore, the resulting combination of cytokines should not be viewed as a unique subset of markers. Other models with the same number of cytokines (not shown in the Results) often led to very similar results. For instance, all of the tested 3-variable models led to very similar classification rates. The large number of possible combinations, and the computational demands of iteratively partitioning the training and test sets, prevented an exhaustive search of all possible models. Our observation that CA-125 had a relatively high specificity for but low sensitivity for early stages ovarian agrees with the published (e.g., Folk et al., Monitoring cancer antigen 125 levels in induction chemotherapy for epithelial ovarian carcinoma and predicting outcome of second-look procedure Gynecol Oncol. 1995 May;57(2):178-82). Interestingly, forcing CA-125 into classification algorithm resulted in worse classification results, that is, lower sensitivity.

Combinations of several serum markers as measured by LabMap technique provided high specificity and sensitivity. The predictive power of combined serological markers for early stage ovarian cancer, as determined by LabMap technology, is thus comparable to that reported by Petricoin and Liotta group for proteomic spectra identified by SELDI-TOF technology (Gyn Oncol 2003). However, when the two techniques are compared, the LabMap assay offers a more reproducible and less expensive approach. To the best of our knowledge, in this study, a highest predictive power was achieved as compared with other publications using serological markers. Table H reflects the available data on sensitivity and specificity of single and combined serum markers.

TABLE H
Sensitivity and specificity of LabMap serum marker panel
vs. published data in detection of early ovarian cancer

| Marker(s) | Sensitivity | Specificity | Reference |
|---|---|---|---|
| CA-125 | | | |
| kallikrein 6 (hK6) | 95 | 47 | Diamandis¹ |
| HK6 + CA-125 | 90 | 42 | |
| HK10 | 90 | 54 | Luo² |
| HK10 + CA-125 | 90 | 70 | |
| SEGFR | | 64 | Baron³ |
| Prostasin | 92 | 94 | Skates⁴ |
| Osteoponin | | | |
| Inhibin | 82 | 54 | Robertson⁵ |
| CA-125 + MCS-F + | 95 | 90 | |
| OVX1 | | | |
| Urinary | 66 | 90 | Nam, Cole⁶ |
| gonadotropin | | | |
| fragment (UGP) | | | |
| VEGF | 54-71 | 65-77 | Oehler et al.; |
| | | | Obermair et al.; |
| | | | Tanir et al.; |
| | | | and Cooper et al.⁷ |
| ¹Diamandis 2002 | | | |
| ²Luo | | | |
| ³Baron et al., 1999. | | | |
| ⁴Skates | | | |
| ⁵Robertson | | | |
| ⁶Nam, Cole | | | |
| ⁷Oehler, M. K. and H. Caffier, Prognostic relevance of serum vascular endothelial growth factor in ovarian cancer. Anticancer Res, 2000. 20(6D): p. 5109-12; Obermair, A., et al., Concentration of vascular endothelial growth factor | | | |
| # (VEGF) in the serum of patients with suspected ovarian cancer. Br J Cancer, 1998. 77(11): p. 1870-4; Tanir et al., Preoperative serum vascular endothelial growth factor (VEGF) in ovarian masses. Eur J Gynaecol Oncol. 2003; 24(3-4): 271-4; and Cooper et al., 2002. | | | |

Interestingly, the reduction in classification rates was observed for models with increasing numbers of cytokines (beyond the optimal model). This phenomenon may be at least partially due to sample size limitations. Although sufficient data were available to obtain very accurate classification, high sensitivity, and high specificity, further model complexity, and more accurate results may be obtained once further data collection allows for larger sample sizes. The general rule of having at least 10 observations per variable (i.e. cytokine in the model) is only approximately satisfied with 2-3 variables in the model. The linear nature of the logistic model may also introduce some limitations, since the probability of cancer may simultaneously depend on the joint combination of multiple cytokines. Future analyses will incorporate other more flexible regression and classification methods such as neural networks and classification trees.

### EXAMPLE 2

### Purification of Circulating Antibodies

Antigen-specific (monospecific) circulating antibodies, or populations of two or more such circulating antibodies can be purified, without limitation, according to the following protocol, thereby facilitating the assays for determining serum concentrations of specific circulating antibodies. The Ig purified in this manner can be used as a control for accurately quantitating individual circulating antibodies.

Purified antigens of interest, for example, IL-6, IL-8, EGF, EGFR, VEGF, Her2/neu, PDGF, PDGFR, survivin, Fas, FasL, CA-125, CA 15-3, CA 19-9, CA 72-4, CEA, MUC-1, PSA; AFP, bHCG (human chorionic gonadotropin), transglutaminase, c-myc, N-Ras, K-Ras, p53; cyclin B, cyclin D, Akt1 (v-akt murine thymoma viral oncogene homolog 1), and others can be covalently coupled to carboxylate-modified polystyrene beads (Cat. No. CLB4, Sigma Chemical Co.) using, without limitation, the above-described protocols for coupling proteins to Luminex beads. For instance, as shown in the Examples below, IL-6 and IL-8 were obtained from Peprotech, Inc., Rocky Hill N.J.; EGF, EGFR, VEGF, Her2/neu, PDGF, PDGFR, survivin, Fas and FasL were obtained from R&D Systems, Inc., Minneapolis, Minn.; CA-125, CA 15-3, CA 19-9, CA 72-4, CEA, MUC-1, PSA; AFP and bhCG were obtained from Fitzgerald Industries International, Inc, Concord, Mass.; transglutaminase was obtained from Sigma-Aldrich Corp., St. Lois, Mo.; c-myc, N-Ras, K-Ras, p53; cyclin B and cyclin D were obtained from Santa Cruz Biotechnology, Inc., Santa Cruz, Calif.; and Akt1 was obtained from Biosource International, Camarillo, Calif. The coupling reaction will be performed in PBS, 5% BSA, 0.01% Tween 20. One mL of beads will couple up to 2 mg of protein. The affinity column will be equilibrated with 5 column volumes of the above-described coupling buffer.

Serum sample diluted, for example and without limitation, 1:2 with PBS will be applied to the column and incubated for 30-60 min at RT (approximately 25° C.). The affinity column will be washed with 15 column volumes of binding buffer. Bound immunoglobulins (approximately 99% IgG/1% IgM) will be eluted with 5 column volumes of the ImmunoPure® IgG Elution Buffer (Cat. No. 21004, Pierce Biotechnology, Inc.). Elution will be monitored by absorbance at 280 nm. Eluate will be neutralized by adding 50 μl of 1 M Tris, pH 9.5 or by adding 100 μl of ImmunoPure® Binding Buffer. During the next step, IgM molecules will be removed using affinity column with Sigma beads covalently coupled to rabbit antibody against human IgM (Jackson ImmunoResearch Laboratories, Inc., West Grove, Pa.). The procedure will be performed exactly as described for primary affinity binding. Finally, protein concentration will be measured by spectrophotometry. If necessary or desirable, thus purified human monospecific IgG preparations can be concentrated, sterilized, aliquoted and frozen for long-term storage according to standard methodology.

### EXAMPLE 3

### Serum Cytokine Analysis

Patient populations. Patient populations are described in Example 1. In this study, fewer samples from each group were utilized (Table I).

TABLE I
Patient characteristics

| Patient Group | Age | Histologic Types |
|---|---|---|
| Control | Range 36-76 | |
| N = 45 | Median 46 | |
| Early Stage | Range 34-88 | Papillary serous carcinoma (n = 13) |
| Ovarian Cancer | Median 46 | Carcinoma, endometroid (n = 10) |
| N = 44 | | Carcinoma, mucinous (n = 7) |
| | | Carcinoma, poorly differeniated (n = 6) |
| | | Adenocarcinoma, serous (n = 5) |
| | | Carcinoma, clear cell (n = 3) |
| Benign Tumors | Range 28-87 | Adenofibroma, serous (n = 1) |
| N = 37 | Median 44.5 | Brenner tumor (n = 1) |
| | | Crystadenofibroma, serous (n = 2) |
| | | Cyst, paratubal (n = 2) |
| | | Cyst, serous (n = 1) |
| | | Cyst, simple (n = 3) |
| | | Cystadenofibroma, serous (n = 3) |
| | | Cystadenoma, mucinous (n = 8) |
| | | Cystadenoma, serous (n = 9) |
| | | Endometriosis (n = 1) |
| | | Fibrosis (n = 1) |
| | | Ovary benign (n = 3) |
| | | Mucinous benign (n = 2) |

Multiplex LabMap tassays for EGF, IL-6, IL-8, G-CSF, VEGF, CA-125 and MCP-1 were performed substantially as described in Example 1. However, each analyte was tested in a single bead assay to determine the optimal concentration of detection antibody. Next, the microspheres were multiplexed and optimized for incubation times and reporter signal. As a reporter signal, streptavidin-PE (Molecular Probes, Inc, Eugene Oreg.) was tested at different concentrations. The minimum cytokine detection levels for EGFR and FasL were <5 pg/ml, and for CA125, <5 IU/ml. Intra-assay variability, expressed as a coefficient of variation, was calculated based on the average for ten patient samples and measured twice at two different time points. The intra-assay variabilities within the replicates presented as an average coefficient of variation were in the range of 5.4-9.1% (data not shown). Inter-assay variability was evaluated by testing quadruplicates of each standard and ten samples. The variabilities of these samples were between 5.6 and 9.6% (data not shown). These single assays were combined in one multiplexed assay and further optimized. Inter-assay variabilities for individual cytokines in 24-plex were in the range of 3.5-9.8% and intra-assay variabilities were in the range of 3.6-12.6% (information provided by Biosource International).

Statistical analysis of data. Descriptive statistics and graphical displays (i.e. dot plots) were prepared to show the distribution of each marker for each disease state. The Wilcoxon rank-sum test, which is the nonparametric equivalent to the t-test, was used to evaluate the significance of differences in marker expression between each disease state. Spearman's (nonparametric) rank correlation was also calculated to quantify the relationships between each pair of markers.

Discrimination of ovarian cancer status was accomplished using classification trees (CART) implemented through S-Plus statistical software. Classification trees discriminate between outcome classes (e.g. cancer patients versus controls) by first searching the range of each potential predictor (e.g. a given cytokine) and finding the split that maximizes the likelihood of the given data set. Within each resulting subset (or node), the algorithm again searches the range of each variable to choose the optimal split. This process is continued until all observations are perfectly discriminated, or the sample size within a given node is too small to divide further (i.e. n=5 or less). Only two observations in the data set had missing values for any of the markers and were excluded from the analysis. The final output of the resulting classification tree is a graphical display of decision criteria for each split and resulting predicted probabilities of being a case across the final splits (i.e. terminal nodes). Several other methods (logistic regression and neural networks) were also implemented with similar, but somewhat less optimal results (results not shown).

Ten-fold cross-validation was implemented to assess classification accuracy using independent data. Specifically, the data were randomly split into ten subsets of equal size (or as equal as possible; nₖ=8-9 for these data). For each subset, a model was fit to the 90% of the data outside that subset; the resulting model (or tree) was then applied to the 10% of data within the given subset. The resulting estimate of classification accuracy therefore utilizes separate subsets of data for model fitting and validation, and thus avoids re-substitution bias. The resulting sensitivity and specificity are reported across a range of decision rules (i.e. cut-points for classifying a given predicted probability as either a case or control) to generate the receiver operator characteristic (ROC) curve. The ROC curve is a graphical display of the sensitivity by (1-specificity) across the different cut-points. Since cross-validation produces a potentially different model for each subset of the data, however, the classification tree produced using all observations (i.e. without cross-validation) was displayed for purposes of describing the optimal model. When not otherwise stated, observations with a predicted probability above 0.5 are classified as a case (or as a benign condition for the comparison of benign versus controls).

Cytokines and CA 125 in Ovarian Cancer Patients

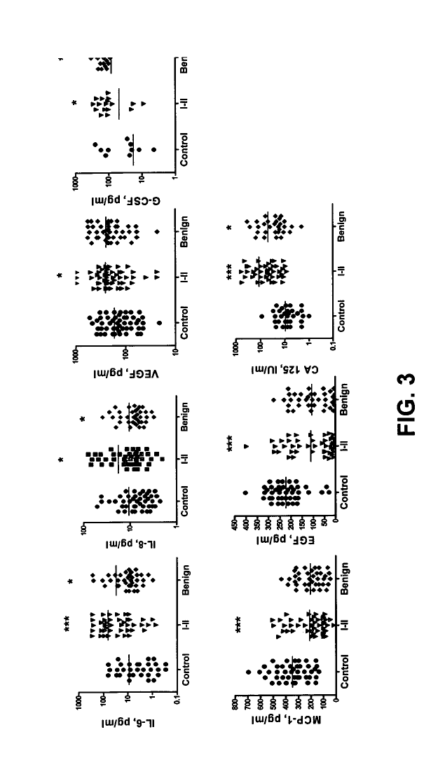

Circulating concentrations of 28 different serum markers belonging to different functional groups were evaluated in a multiplexed assay using LabMAP™ technology, in serum samples of patients from three clinical groups: women with early (I-II) stage ovarian cancer, women with benign pelvic masses, and age-matched healthy controls (Table I). Serum levels of IL-2, IL-4, IL-5, IL-10, IL-13, IL-15, IL-17, IL-18, TNFα, IFNγ, and survivin were undetectable in either control or patients' sera. IL-1β, IL-12p40, MIP-1α, MIP-1β, HGF, RANTES, bFGF, GM-CSF, TGFβ demonstrated measurable serum concentrations, which did not differ between the control and patient groups (data not shown). Serum concentrations of IL-6, IL-8, G-CSF, C A125, and VEGF were found to be significantly higher in ovarian cancer patients as compared to controls (P<0.05-P<0.001) (Table J and FIG. 3). LabMAP™ assays demonstrated relatively high serum concentrations of EGF (224±12 pg/ml) and MCP-1 (384±21 pg/ml) (Table J and FIG. 3). Surprisingly, serum levels of EGF and MCP-1 were significantly (P<0.05-P<0.001) lower in ovarian cancer patients as compared to controls (Table I and FIG. 3).

TABLE J
Levels of serum markers

| Analytes/Patients | | Healthy Controls | Ovarian Cancer | Benign |
|---|---|---|---|---|
| EGF | Mean ± SE | 223.8 ± 11.46 | 110.7 ± 15.58*** | 98.6 ± 12.35*** |
| | Median (Range) | 238 (29.8-402.6) | 74.9 (0-396.9) | 94.9 (0-276.4) |
| IL-6 | Mean ± SE | 8.8 ± 2.50 | 64.2 ± 12.72*** | 28.0 ± 9.3*** |
| | Median (Range) | 0 (0-64.1) | 23.8 (0-280.2) | 7.6 (0-275.3) |
| G-CSF | Mean ± SE | 21.8 ± 8.44 | 49.2 ± 12.04^{NS} | 77.4 ± 14.04** |
| | Median (Range) | 0 (0-257.6) | 0 (0-290.8) | 0 (0-339.1) |
| IL-8 | Mean ± SE | 10.2 ± 1.68 | 24.0 ± 5.98** | 12.4 ± 3.11 |
| | Median (Range) | 6 (2.3-51.4) | 9.6 (2.0-180.6) | 7.6 (3.0-127.8) |
| VEGF | Mean ± SE | 90.7 ± 10.52 | 153.5 ± 19.95* | 258.8 ± 26.04* |
| | Median (Range) | 67 (18-306) | 106 (28-552) | 218 (48-662) |
| CA-125 | Mean ± SE | 10.4 ± 2.28 | 153.7 ± 44.04*** | 51.8 ± 13.23** |
| | Median (Range) | 6.0 (0-87) | 51.0 (0-1412) | 16.0 (0-372) |
| MCP-1 | Mean ± SE | 341.8 ± 21.34 | 210.3 ± 20.54*** | 196.3 ± 16.06*** |
| | Median (Range) | 326.8 (135.5-695.7) | 172.9 (17.1-502.3) | 178.2 (44.9-434.6) |

Comparison of ovarian cancer or benign patients with controls
*P < 0.05;
**P < 0.01;
***P < 0.001

Specifically, FIG. 3 shows serum levels of cytokines and growth factors in healthy controls, ovarian cancer patients at stages I-II and patients with benign gynecological disease. Sera were collected from 45 patients with early stage (I-II) ovarian cancer, 44 patients with benign pelvic masses and from 37 age and sex-matched healthy controls. Circulating concentrations of cytokines and growth factors were measured using LabMAP technology as described in Methods. Measurements were performed twice. Horizontal lines indicate mean values. * denotes statistical significance between controls and cancer patients of p<0.05; **−p<0.01; ***−p<0.001.

Serum of patients with benign tumors had elevated levels of VEGF, G-CSF and CA-125 as compared to controls (P<0.05). However, no statistical differences were observed for G-CSF and VEGF concentrations between cancer and benign groups. CA-125 levels were significantly (P<0.05) lower in the benign group as compared to the cancer group. Patients with benign tumors were characterized to have lower levels of EGF, IL-12p40 and MCP-1 (Table J and FIG. 3). However, circulating concentrations of IL-6 and IL-8 were elevated only in the sera of ovarian cancer patients but not in benign cases (Table J and FIG. 3).

Statistical analysis of serum cytokines as ovarian cancer biomarkers—Comparison of early stage ovarian cancer vs. healthy controls. Table J illustrates classification results using each individual cytokine to distinguish early stage ovarian cancer from controls. Results show that the individual markers led to only moderately accurate prediction of early stage cancer. Only CA-125, EGF and IL-6 correctly classified over 80% of the test set subjects (Table K).

TABLE K
Predictive values for single serum markers
for early stage ovarian cancer

| | % Correctly | | |
|---|---|---|---|
| Cytokine | Classified | Sensitivity | Specificity |
| CA125 | 85.1 | 95.5 | 74.4 |
| IL-6 | 85.1 | 84.1 | 86.0 |
| EGF | 80.5 | 84.1 | 76.7 |
| IL-8 | 79.3 | 88.6 | 69.8 |
| MCP | 78.2 | 84.1 | 72.1 |
| VEGF | 73.6 | 79.5 | 67.4 |
| G-CSF | 73.6 | 72.7 | 74.4 |

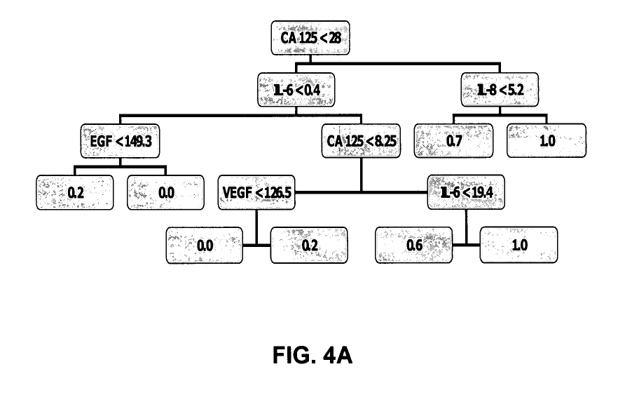

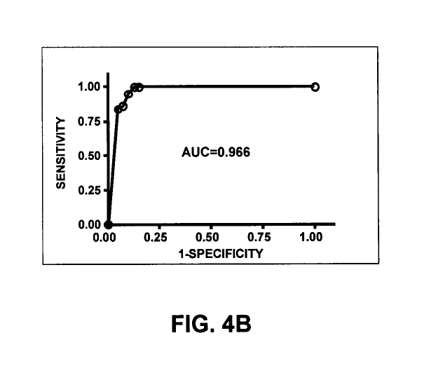

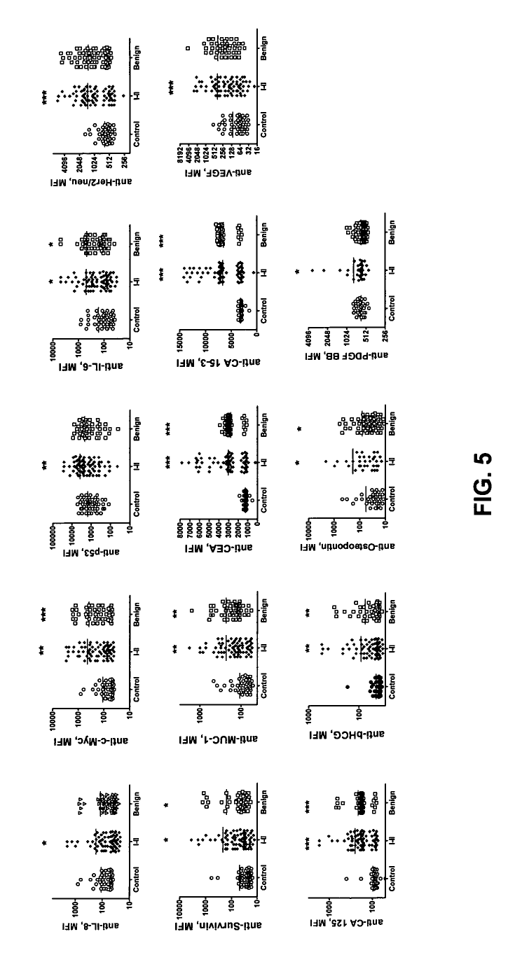

FIG. 4A displays the classification tree using CART methodology for discriminating controls from early stage ovarian cancer. The model in FIG. 3 utilized all observations in either group to fit the model (as opposed to cross-validation, which is utilized for subsequent estimation of classification accuracy as explained in subsequent paragraphs). The classification tree utilized five of the eight markers, including CA125, EGF, VEGF, IL-6, and IL-8. The range of data specified at each split (e.g. CA-125<26) represents the subset of data which is further subdivided by branches to the left. For example, subjects with CA-125<26 were then further subdivided by IL-6 (<6.35 versus >6.35), whereas subjects with CA-125>26 were then further subdivided by levels of IL-8 (<5.265 versus >5.165). The numbers specified for each of the final groups (i.e. terminal nodes) represent the probability of being a case within each subset.

Rates of classification accuracy (in discriminating controls from early stage cancer) were then obtained using 10-fold cross-validation. FIG. 4B displays the resulting ROC curve. As described in the Methods section, the sensitivity and specificity depend on the cut-point (i.e. predicted probability from the classification tree) used to classify each subject as either a case or control. Using the standard cut-point of 0.5 (i.e. everyone with a predicted probability above 0.5 is classified as a cancer case) gives 100% sensitivity, 86% specificity, and 93% correctly classified. Fixing the specificity at 91% still leads to a very high sensitivity, at 95.5% (again with 93% correctly classified). Alternatively, a specificity of 95.3% corresponds to a sensitivity of 84.1% (and 90.0% correctly classified). The total area under the receiver operating characteristic (ROC) curve was near one (which would represent perfect classification), at 0.966.

Specifically, FIG. 4A provides a classification tree for discriminating early stage ovarian cancer from healthy controls. Rectangles represent splitting nodes containing cytokine and cytokine cut-off. The range of data specified at each split represents the subset of data which is further subdivided by branches to the left. The numbers specified for each of the final groups (i.e. terminal nodes) represent the probability of being a case within each subset. FIG. 4B provides a Receiver Operating Characteristic (ROC) curve for biomarker panel. Presented are results from 10-fold cross validation of classification tree analysis of early stage ovarian cancer versus healthy controls.

Several models provided comparable high sensitivity and specificity for early diagnosis of ovarian cancer. Therefore, the resulting combination of cytokines should not be viewed as a unique subset of markers. Other models with the same number of cytokines (not shown), often led to very similar results. For instance, all of the tested 3-variable models led to very similar classification rates. The large number of possible combinations, and the computational demands of iteratively partitioning the training and test sets, prevented an exhaustive search of all possible models.

Comparison of controls and early stage ovarian cancer vs. benign conditions. To assess the validity of serum biomarker panel for discrimination of benign pelvic tumors from the other groups, separate classification tree models were fit to predict 1) benign conditions versus early stage cancer, and 2) benign conditions versus controls. The same 10-fold cross-validation procedure was utilized to assess classification accuracy. For the comparison of benign versus cancer, 80.2% of subjects were correctly classified, with a sensitivity of 84.1% and a specificity of 75.7%. The classification tree for comparison of benign versus cancer (not shown) utilized five markers, (CA125, G-CSF, IL-6, EGF, and VEGF). For the comparison of benign versus controls, 90.0% of subjects were correctly classified, with a sensitivity of 86.5% and a specificity of 93.0%. The classification tree for comparison of benign versus controls (not shown) utilized six of the eight markers, including EGF, VEGF, G-CSF, CA125, IL-6, and IL-8.

### EXAMPLE 4

### Development of LabMAP Assays for Circulating Antibodies

Assays were performed in filter-bottom 96-well microplates (Millipore). Purified antigens of interest (IL-6, IL-8, EGF, EGFR, VEGF, Her2/neu, PDGF, PDGFR, survivin, Fas, FasL, CA-125, CA 15-3, CA 19-9, CA 72-4, CEA, MUC-1, PSA; AFP, bhCG, transglutaminase, c-myc, N-Ras, K-Ras, p53; cyclin B, cyclin D and Akt1, sources described in Example 2) were coupled to Luminex beads as described for antibodies. Antigen-coupled beads were pre-incubated with blocking buffer containing 4% BSA for 1 h at room temperature on microtiter shaker. Beads were then washed three times with washing buffer (PBS, 1% BSA, 0.05% Tween 20) using a vacuum manifold followed by incubation with 50 μl blood serum diluted 1:250 for 30 min at 4° C. This dilution was selected as an optimal for recovery of anti-IL-8 IgG based on previous serum titration (data not shown). Next, washing procedure was repeated as above and beads were incubated with 50 μl/well of 4 μg/ml PE-conjugated donkey antibody raised against human IgG (Jackson Laboratories), for 45 min in the dark with the constant shaking. Wells were washed twice, assay buffer was added to each well and samples were analyzed using the Bio-Plex suspension array system (Bio-Rad Laboratories, Hercules, Calif.). For standard curve, antigen-coupled beads were incubated with serially diluted human antibodies against specific antigens. Purification of monospecific human antibodies is described above. Data analysis was performed using five-parametric-curve fitting.

### EXAMPLE 5

### LabMAP Analysis of Circulating Antibodies in Patients with Early Stage Ovarian Cancer, Patients with Benign Pelvic Masses and Control Healthy Women

A panel was generated for analysis of circulating antibodies. This panel includes 28 assays for the following antibodes: IL-6, IL-8, EGF, EGFR, VEGF, Her2/neu, PDGF, PDGFR, CA-125, CA 15-3, CA 19-9, CA 72-4, CEA, MUC-1, PSA, AFP, bhCG, survivin, Fas, FasL, transglutaminase, c-myc, N-Ras, K-Ras, Akt1, p53, cyclin B, cyclin D. To quantitate the results, standard curve of purified human IgG was utilized. For accurate quantitation, human antibodies specific to a given antigen (monospecific) were purified from blood serum as described above in Example 2. The serum samples we the samples described above in Example 1 plus an additional 31 samples from patients with early stages ovarian cancer, 60 samples from patients with benign condition (Table A), and 30 additional control samples were analyzed. Serum concentrations of antibodies against following twelve antigens were found to be significantly higher in ovarian cancer patients as compared to controls and patients with benign pelvic masses (P<0.05-P<0.001), IL-6, IL-8, c-myc, p53, CA-125, CEA, CA 15-3, MUC-1, survivin, bHCG, osteopontin, PDGF BB (FIG. 3).

Comparison of early stage ovarian cancer vs. healthy controls. The classification tree utilized five of the thirteen markers, including CA15-3, IL-8, survivin, p53, c-myc. Using the standard cut-point of 0.5 gives 95% sensitivity, 100% specificity, and 98% correctly classified. Other combinations of three to about eight of the above twelve circulating antibodies also offered high classification results.

Comparison of controls and early stage ovarian cancer vs. benign conditions. As shown in Table L for the comparison of benign versus cancer, 89% of subjects were correctly classified, with a sensitivity of 95% and a specificity of 80%. The classification tree for comparison of benign versus cancer (not shown) utilized antibodies against following eight antigens, CA 15-3, CEA, IL-6, IL-8, p53, c-myc, bHCG and survivin. For the comparison of benign versus controls, 98% of subjects were correctly classified, with a sensitivity of 96% and a specificity of 99%. The classification tree for comparison of benign versus controls (not shown) utilized four markers, including CA 15-3, IL-8, MUC1 and c-myc.

TABLE L
Diagnostic power of multiplexed antibody assay

| | Markers included | | | |
|---|---|---|---|---|
| | in the | % Correctly | | |
| Comparison | classification tree | Classified | Sensitivity | Specificity |
| Control vs. | CA15-3, IL-8, | 98% | 95% | 100% |
| Early Stage | survivin, p53, | | | |
| | c-myc | | | |
| Benign vs. | CA15-3, CEA, | 89% | 95% | 80% |
| Early Stage | p53, IL-6, | | | |
| | c-myc, bHCG, | | | |
| | IL-8, survivin | | | |
| Control vs. | CA 15-3, IL-8, | 98% | 96% | 99% |
| Benign | MUC1, c-myc | | | |

### EXAMPLE 6

### Generation of LabMAP Assays for Cancer Markers

Assays for ErbB2, CA 15-3, CEA, Fas, FasL, EGFR, CA-125, cytokeratin 19 (Cyfra 21-1), kallikrein-8, M-CSF (macrophage colony stimulating factor) were developed as described in Example 1. The sources of antibodies and standards used for development of these assays are presented in Table M.

TABLE M
Source of reagents for development of the Cancer Markers panel

| Target | Antigen | Capture | Detect |
|---|---|---|---|
| ErbB2 | R&D Systems | R&D Systems | R&D Systems |
| CA15-3 | Fitzgerald | Biodesign | Fitzgerald |
| CEA | Fitzgerald | Fitzgerald | Fitzgerald |
| FasL | Peprotech | MBL | R&D Systems |
| EGFR | R&D Systems | R&D Systems | R&D Systems |
| CA125 | Fitzgerald | Fitzgerald | Fitzgerald |
| Cytokeratin 19 | Calbiochem | Progen | Progen |
| Fas | R&D Systems | R&D Systems | R&D Systems |
| Her2/neu | R&D Systems | R&D Systems | R&D Systems |
| kallikrein-8* | R&D Systems | R&D Systems | R&D Systems |
| M-CSF | R&D Systems | R&D Systems | R&D Systems |

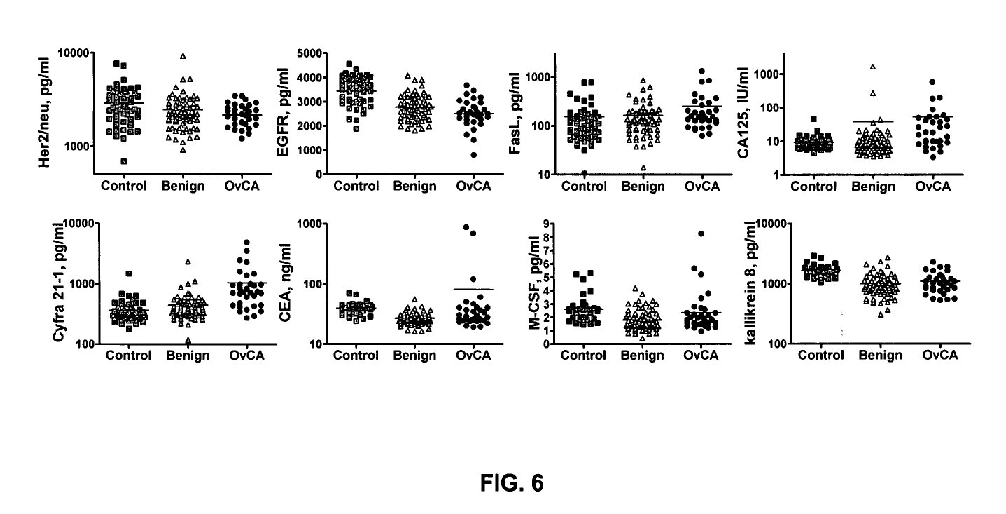

LabMAP analysis cancer markers in patients with early stage ovarian cancer, patients with benign pelvic masses and control healthy women. For this project, 31 samples from patients with early stages ovarian cancer, 60 samples from patients with benign condition, and 30 additional control samples (included in Table A) were utilized. Serum concentrations of CA-125 and Cyfra 21-1 were found to be significantly higher in ovarian cancer patients as compared to controls and patients with benign pelvic masses (P<0.05-P<0.001). Concentrations of Her2/neu and EGFR were significantly (P<0.05) lower in cancer group than in the control and benign groups (FIG. 6).

Comparison of early stage ovarian cancer vs. healthy controls. The following data were generated using statistical machine learning to optimize the unbiased performance of algorithms for predicting the masked class labels of LUMINEX profiles. This naïve Bayes analysis resulted in 91% sensitivity, 94% specificity, and 92% correctly classified.

Comparison of controls and early stage ovarian cancer vs. benign conditions. For the comparison of benign versus cancer using the combination of these four markers, 76% of subjects were correctly classified, with a sensitivity of 40% and a specificity of 94%. For the comparison of benign versus control using the combination of these four markers, 87% of subjects were correctly classified, with a sensitivity of 86% and a specificity of 89%.

Data also was analyzed using the CART program, the results of which are shown in tables N and O. Using the panel cytokeratin 19, kallikrein 8, CEA, CA 125, M-CSF to distinguish cancer vs. controls resulted in 94% sensitivity, 94.0% specificity and 94% correctly classified. Other useful panels include: 1) cytokeratin-19, CEA, CA-125, M-CSF and EGFR; 2) cytokeratin-19, kallikrein-8, CA-125, M-CSF and Fas; 3) cytokeratin-19, kallikrein-8, CEA and M-CSF; and 4) cytokeratin-19, kallikrein-8, CEA and CA-125. Using the panel CA 125, cytokeratin 19, ErbB2 to evaluating cancers vs. benign growths using CART methodology, 85.9% of subjects were correctly classified, with a sensitivity of 81.3% and a specificity of 88.1%.

TABLE N
Analysis of Cancer versus Benign

| Markers Found in the Tree | Classification | | |
|---|---|---|---|
| Model | Rate | Sensitivity | Specificity |
| CA 125, cytokeratin 19, | 85.9% | 81.3% | 88.1% |
| ErbB2 | | | |
| CA-125, CK-19, Fas, M-CSF | 87.9% | 78.1% | 92.5% |
| CK-19, CEA, Fas, EGFR, | 81.8% | 75.0% | 85.1% |
| kallikrein-8 | | | |
| CEA, Fas, M-CSF, EGFR, | 85.8% | 84.4% | 86.6% |
| CA-125 | | | |

TABLE O
Analysis of Cancer versus Controls

| Markers Found in the Tree | Classification | | |
|---|---|---|---|
| Model | Rate | Sensitivity | Specificity |
| cytokeratin 19, kallikrein 8, | 93.9% | 93.8% | 94.0% |
| CEA, CA 125, M-CSF | | | |
| kallikrein-8, EGFR, CA-125 | 89.0% | 90.6% | 88.0% |
| CK-19, CEA, CA-125, M-CSF, | 86.6% | 81.3% | 90.0% |
| EGFR | | | |
| CK-19, kallikrein-8, CA-125, | 91.5% | 84.4% | 96% |
| M-CSF, Fas | | | |
| CK-19, kallikrein-8, CEA, M- | 90.2% | 84.4% | 94% |
| CSF | | | |
| CK-19, kallikrein-8, CEA, CA- | 90.2% | 93.8% | 88.0% |
| 125 | | | |

### EXAMPLE 7

### Longitudinal Study

A multimodal randomized control trial (RCT) was performed in St Bartholomew's Hospital, London, UK, started in 1996, with annual screening ending in December 2001 and follow up for cancer through to December 2003 (Skates, S J et al. Calculation of the Risk of Ovarian Cancer from Serial CA-125 Values for Preclinical Detection in Postmenopausal Women J. Clin. Oncol. 2003 21(Suppl.):206-210; “Skates et al.”). This trial is a unique serum based ovarian cancer screening trial using CA125 and the ‘Risk of Ovarian Cancer’ algorithm described in Skates et al. The study was undertaken to prospectively evaluate the algorithm and to determine the feasibility of such an RCT in the UK. In the trial, 13,688 postmenopausal women, over 50 years of age (self referred) were recruited. Baseline epidemiological information was obtained on all women and 6734 were randomized to the screen arm. These women underwent annual screening for 2-6 years. Screening ended in December 2001. Serial samples at intervals of 6 weeks to one year over six years were available. A total of 35,175 samples are available in the serum bank and follow-up to document the incidence of cancers and other common diseases is in progress. The most unique and precious samples from this collection are the preclinical samples from women diagnosed to have ovarian cancer. The serum bank from the study currently includes a set of 93 serum samples from 19 women dating from <1 to 6 years prior to the development of ovarian/fallopian tube cancer detection by screening as opposed to symptomatic presentation.

All cases and controls are women aged ≧50, postmenopausal with no high risk family history—all have 1 or no relatives with ovarian cancer. Each serum sample from a study participant diagnosed with primary ovarian/fallopian tube cancer was matched with 3 samples from women who remained healthy. All samples were taken and transported in clotted tubes at room temperature by the post. On reaching the central laboratory, they were immediately spun and separated and the serum was stored in freezers at −20° C. Sample transit time was recorded for all samples. All had a transit time of less than 56 hours. For the current study, one aliquot of the sample was thawed and distributed into 100 mL aliquots, which were stored in −20° C. freezers.

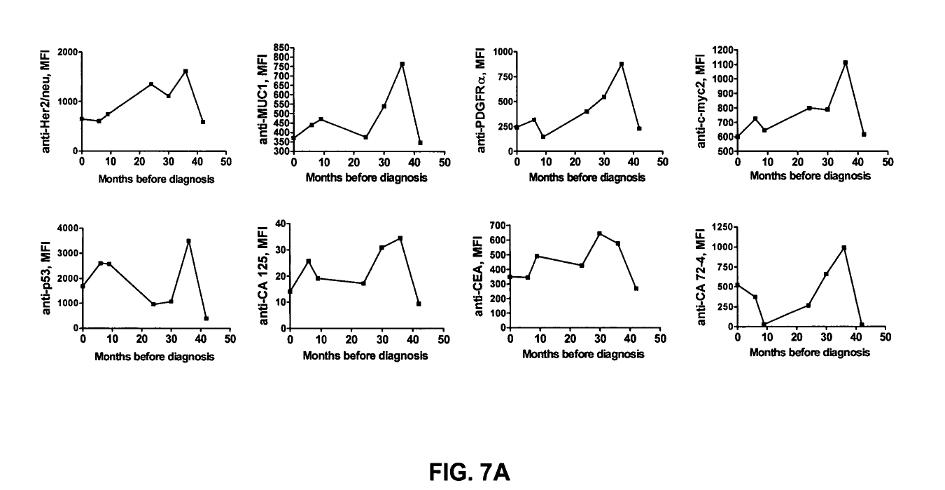

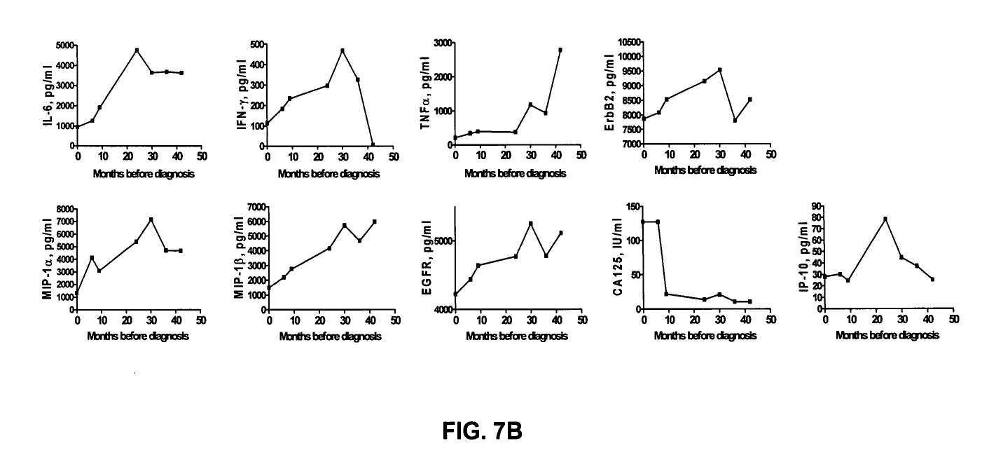

Serial serum samples from women on Bart's study who developed ovarian cancer, were analyzed using LabMAP technology for cytokines, circulating antibodies and cancer markers described in Examples 1-6. FIGS. 7A and 7B demonstrate transient increase in concentrations (averaged among 11 patients) of antibodies against Her2/neu, MUC-1, c-myc, p53, CA-125, CEA, CA 72-4, PDGFRα (FIG. 7A), and of cytokines, IL-6, IP-10 (interferon gamma-inducible protein, MW 10 kDa) and IFNγ about 30-40 months before diagnosis. Furthermore, concentrations of TNFα, MIP-1α, MIP-1β, EGFR and Her2/neu steadily decrease starting as of 40 months prior to diagnosis (FIG. 7B). Increase in average CA-125 concentration can be visible only 9 months prior to diagnosis (FIG. 7B). Moreover, at present increasing of CA-125 does not present enough justification for intervention. Therefore, combination of velocities of several markers might serve as a sufficient indication of ovarian carcinogenesis for surgical intervention. In FIGS. 7A and 7B, for 3-30 months points, n=11; 36 months actually represents a time period from 36 to 42 months (n=11), 42 months actually represents a time period of 42 months and greater (n=9).

Multiplex Luminex LabMAP assays were performed essentially as described above in Examples 1 and 3 for circulating proteins IL-6, IFN-γ, GM-CSF, TNFα, MCP-1, MIP-1α, MIP-1β, bFGF, HGF, IP-10, IL-12p40, IL-15, CEA, ErbB2 and EGFR and for circulating antibodies anti-EGF, anti-IL-8, anti-VEGF, anti-p53, anti-survivin, anti-Her2/neu (human epidermal growth factor receptor 2), anti-MUC1, anti-c-myc, anti-c-myc2, anti-osteopontin, anti-PSA, anti-CA-125, anti-CEA, anti-CA 72-4, anti-PDGF, anti-Akt1, and anti-PDGFRα (platelet derived growth factor receptor α), as described above in Examples 4 and 5. Circulating antibodies were affinity purified using a mixture of antigen-bound beads as described in Example 2. The antigen-bound beads were prepared in the manner described in Example 2.

Whereas particular embodiments of the invention have been described herein for the purpose of illustrating the invention and not for the purpose of limiting the same, it will be appreciated by those of ordinary skill in the art that numerous variations of the details, materials and arrangement of parts may be made within the principle and scope of the invention without departing from the invention as described in the appended claims.

1. A method of determining the presence of ovarian cancer in a patient, comprising determining levels of markers in a blood marker panel comprising two or more of EGF, G-CSF, IL-6, IL-8, CA-125, VEGF, MCP-1, anti-IL6, anti-IL8, anti CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu in a sample of the patient's blood, where the presence of two or more of the following conditions indicates the presence of ovarian cancer in the patient: EGF_{LO}, G-CSF_{HI}, IL-6_{HI}, IL-8_{HI}, VEGF_{HI}, MCP-1_{LO}, anti-IL-6_{HI}, anti-IL-8_{HI}, anti-CA-125_{HI}, anti-c-myc_{HI}, anti-p53_{HI}, anti-CEA_{HI}, anti-CA 15-3_{HI}, anti-MUC-1_{HI}, anti-survivin_{HI}, anti-bHCG_{HI}, anti-osteopontin_{HI}, anti-Her2/neu_{HI}, anti-Akt1_{HI}, anti-cytokeratin 19_{HI} and anti-PDGF_{HI}, CA-125_{HI}, cytokeratin 19_{HI}, EGFR_{LO}, Her2/neu_{LO}, CEA_{HI}, FasL_{HI}, kallikrein-8_{LO}, ErbB2_{LO} and M-CSF_{LO}.

2. The method of claim 1, wherein the panel comprises 3 to 5 of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, EGFR, CEA, kallikrein-8, M-CSF and Her2/neu.

3. The method of claim 1, wherein the panel comprises 4 of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, EGFR, CEA, kallikrein-8, M-CSF and Her2/neu.

4. The method of claim 1, wherein the panel comprises 5 of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, EGFR, CEA, kallikrein-8, M-CSF and Her2/neu.

5. The method of claim 1, wherein the panel comprises cytokeratin 19.

6. The method of claim 5, wherein the panel further comprises kallikrein-8.

7. The method of claim 6, wherein the panel further comprises CEA.

8. The method of claim 7, wherein the panel further comprises one or both of M-CSF and CA-125.

9. The method of claim 6, wherein the panel further comprises CA-125.

10. The method of claim 9, wherein the panel further comprises one or both of M-CSF and FasL.

11. The method of claim 1, wherein the panel comprises CA-125.

12. The method of claim 11, wherein the panel further comprises CK-19.

13. The method of claim 1, wherein the panel is one of:
a. CA-125, cytokeratin-19, Fas, M-CSF;
b. cytokeratin-19, CEA, Fas, EGFR, kallikrein-8;
c. CEA, Fas, M-CSF, EGFR, CA-125;
d. cytokeratin 19, kallikrein 8, CEA, CA 125, M-CSF;
e. kallikrein-8, EGFR, CA-125;
f. cytokeratin-19, CEA, CA-125, M-CSF, EGFR;
g. cytokeratin-19, kallikrein-8, CA-125, M-CSF, Fas;
h. cytokeratin-19, kallikrein-8, CEA, M-CSF;
i. cytokeratin-19, kallikrein-8, CEA, CA-125;
j. CA 125, cytokeratin 19, ErbB2; and
k. anti-CA 15-3, anti-IL-8, anti-survivin, anti-p53 and anti c-myc.

14. The method of claim 1, further comprising comparing the levels of the two or more markers in the patient's blood with levels of the same markers in a control sample by applying a statistical method selected from the group consisting of linear regression analysis, classification tree analysis and heuristic naïve Bayes analysis.

15. The method of claim 14, wherein the statistical method is performed by a computer process.

16. The method of claim 14, wherein the statistical method is a classification tree analysis.

17. The method of claim 14, wherein the panel generates a sensitivity of at least about 80% and a specificity of at least about 80% using the statistical method.

18. The method of claim 17, wherein the panel generates a sensitivity of at least about 85% using the statistical method.

19. The method of claim 17, wherein the panel generates a specificity of at least about 85% using the statistical method.

20. The method of claim 17, wherein the panel generates a specificity of at least about 90% using the statistical method.

21. The method of claim 17, wherein the panel generates a specificity of at least about 99% using the statistical method.

22. The method of claim 1, wherein the panel comprises two or more of anti-c-myc, anti-p53, anti-CEA, anti-CA 15-3, anti-MUC-1, anti-survivin, anti-bHCG, anti-osteopontin and anti-PDGF.

23. The method of claim 1, wherein the panel comprises two or more of CA-125, cytokeratin 19, EGFR, kallikrein-8, M-CSF, FasL, CEA, and Her2/neu.

24. The method of claim 1, wherein the panel comprises Her2/neu, EGFR, CA-125 and cytokeratin 19.

25. The method of claim 1, wherein the panel comprises anti-CA15-3, IL-8, survivin, anti-p53 and anti-c-myc.

26. The method of claim 1, wherein the panel comprises anti-CA15-3, anti-CEA, anti-IL-6, anti-IL-8, anti-survivin, anti-p53, anti-bHGC and anti-c-myc.

27. The method of claim 1, wherein the blood sample is a serum sample.

28. The method of claim 1, comprising performing an immunoassay to determine the quantities of the two or more of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, anti-Her2/neu, cytokeratin 19, EGFR, CEA, kallikrein-8, M-CSF, FasL, ErbB2 and Her2/neu in the patient's blood.

29. The method of claim 28, wherein the immunoassay utilizes an array comprising binding reagents types specific to EGF, G-CSF, IL-6, IL-8, VEGF and MCP-1, wherein each binding reagent type is attached independently to a one or more discrete locations on one or more surfaces of one or more substrates.

30. The method of claim 29, wherein the substrates are beads comprising an identifiable marker, wherein each binding reagent type is attached to a bead comprising a different identifiable marker than beads to which a different binding reagent type is attached.

31. The method of claim 30, wherein the identifiable marker comprises a fluorescent compound.

32. The method of claim 30, wherein the identifiable marker comprises a quantum dot.

33. An array comprising binding reagent types specific to any two or more of EGF, G-CSF, IL-6, IL-8, CA-125, VEGF, MCP-1, anti-IL6, anti-IL8, anti CA-125, anti-c-myc, anti-p53, anti-CEA, anti-CA 15-3, anti-MUC-1, anti-survivin, anti-bHCG, anti-osteopontin, anti-PDGF, anti-Her2/neu, anti-Akt1, anti-cytokeratin 19, cytokeratin 19, EGFR, CEA, kallikrein-8, M-CSF, FasL, ErbB2 and Her2/neu, wherein each binding reagent type is attached independently to one or more discrete locations on one or more surfaces of one or more substrates.

34. The array of claim 33, wherein the substrates are beads comprising an identifiable marker, wherein each binding reagent type is attached to a bead comprising a different identifiable marker than beads to which a different binding reagent is attached.

35. The array of claim 34, wherein the identifiable marker comprises a fluorescent compound.

36. The array of claim 34, wherein the identifiable marker comprises a quantum dot.

37. The array of claim 33, consisting essentially of binding reagent types independently specific to any two or more of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, EGFR and Her2/neu, each binding reagent type is attached independently to one or more discrete locations on one or more surfaces of one or more substrates.

38. The array of claim 33, wherein the panel comprises 3 to 5 of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, EGFR, CEA, kallikrein-8, M-CSF and Her2/neu.

39. The array of claim 33, wherein the panel comprises 4 of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, EGFR, CEA, kallikrein-8, M-CSF and Her2/neu.

40. The array of claim 33, wherein the panel comprises 5 of EGF, G-CSF, IL-6, IL-8, CA-125, VEGF, MCP-1, anti-c-myc, anti-p53, anti-CEA, anti-CA 15-3, anti-MUC-1, anti-survivin, anti-bHCG, anti-osteopontin, anti-PDGF, cytokeratin 19, EGFR, CEA, kallikrein-8, M-CSF and Her2/neu.

41. The array of claim 33, wherein the panel comprises cytokeratin 19.

42. The array of claim 41, wherein the panel further comprises kallikrein-8.

43. The array of claim 42, wherein the panel further comprises CEA.

44. The array of claim 43, wherein the panel further comprises one or both of M-CSF and CA-125.

45. The array of claim 42, wherein the panel further comprises CA-125.

46. The array of claim 45, wherein the panel further comprises one or both of M-CSF and FasL.

47. The array of claim 33, wherein the panel comprises CA-125.

48. The array of claim 47, wherein the panel further comprises CK-19.

49. The array of claim 33, wherein the panel is one of:
a. CA-125, cytokeratin-19, Fas, M-CSF;
b. cytokeratin-19, CEA, Fas, EGFR, kallikrein-8;
c. CEA, Fas, M-CSF, EGFR, CA-125;
d. cytokeratin 19, kallikrein 8, CEA, CA 125, M-CSF;
e. kallikrein-8, EGFR, CA-125;
f. cytokeratin-19, CEA, CA-125, M-CSF, EGFR;
g. cytokeratin-19, kallikrein-8, CA-125, M-CSF, Fas;
h. cytokeratin-19, kallikrein-8, CEA, M-CSF;
i. cytokeratin-19, kallikrein-8, CEA, CA-125;
j. CA 125, cytokeratin 19, ErbB2; and
k. anti-CA 15-3, anti-IL-8, anti-survivin, anti-p53 and anti c-myc.

50. A method of determining the presence of ovarian cancer in a patient, comprising determining levels of at least one of anti-Her2/neu, anti-IL-8, anti-osteopontin, anti-VEGF and anti-PDGF in a sample of the patient's blood, where the presence of one or more of the following conditions indicates the presence of ovarian cancer in the patient: anti-Her2/neu_{HI}, anti-IL-8_{HI}, anti-osteopontin_{HI}, anti-VEGF_{HI}, and anti-PDGF_{HI}.

51. A method of determining the presence of ovarian cancer in a patient, comprising determining levels of markers in a blood marker panel comprising anti-CA 15-3, anti-IL-8, anti-survivin, anti-p53 and anti c-myc in a sample of the patient's blood, wherein the presence of the following conditions indicates the presence of ovarian cancer in the patient: anti-CA 15-3_{HI}, anti-IL-8_{HI}, anti-survivin_{HI}, anti-p53_{HI}, and anti-c-myc_{HI}.

52. The method of claim 51, wherein the blood marker panel further comprises anti-CEA, anti-IL-6, anti-EGF and anti-bHCG.

53. The method of claim 51, further comprising comparing the levels of the markers in the patient's blood with levels of the same markers in a control sample by applying a statistical method selected from the group consisting of linear regression analysis, classification tree analysis and heuristic naïve Bayes analysis.

54. The method of claim 53, wherein the statistical method is performed by a computer process.

55. The method of claim 53, wherein the statistical method is a classification tree analysis.

56. The method of claim 51, wherein the panel generates a sensitivity of at least about 90% and a specificity of at least about 99% using the statistical method.

57. The method of claim 51, wherein the panel generates a sensitivity of at least about 90% and a specificity of at least about 99% using the statistical method.

58. The method of claim 51, comprising performing an immunoassay to determine the quantities of anti-CA 15-3_{HI}, anti-IL-8_{HI}, anti-survivin_{HI}, anti-p53_{HI} and anti-c-myc_{HI} in the patient's blood.

59. The method of claim 58, wherein the immunoassay utilizes an array comprising binding reagents types specific to EGF, G-CSF, IL-6, IL-8, VEGF and MCP-1, wherein each binding reagent type is attached independently to a one or more discrete locations on one or more surfaces of one or more substrates.

60. The method of claim 59, wherein the substrates are beads comprising an identifiable marker, wherein each binding reagent type is attached to a bead comprising a different identifiable marker than beads to which a different binding reagent type is attached.

61. The method of claim 60, wherein the identifiable marker comprises a fluorescent compound.

62. The method of claim 60, wherein the identifiable marker comprises a quantum dot.

63. An array comprising binding reagent types specific to anti-CA 15-3, anti-IL-8, anti-survivin, anti-p53 and anti c-myc, wherein each binding reagent type is attached independently to one or more discrete locations on one or more surfaces of one or more substrates.

64. The array of claim 63, further comprising binding reagent types specific to anti-CEA, anti-IL-6, anti-EGF and anti-bHCG, wherein each binding reagent type is attached independently to one or more discrete locations on one or more surfaces of one or more substrates.

65. A method of predicting onset of clinical ovarian cancer comprising determining the change in concentration at two or more time points of two or more of anti-Her2/neu, anti-MUC-1, anti-c-myc, anti-p53, anti-CA-125, anti-CEA, anti-CA 72-4, anti-PDGFRa, IFNγ, IL-6, IL-10, TNFα, MIP-1α, MIP-1β, EGFR and Her2/neu in a patient's blood, wherein an increase in the concentration of anti-Her2/neu, anti-MUC-1, anti-c-myc, anti-p53, anti-CA-125, anti-CEA, anti-CA 72-4, anti-PDGFRα, IFNγ, IL-6 and IL-10 in the patient's blood between the two time points and a decrease in the concentration of TNFα, MIP-1α, MIP-1, EGFR and Her2/neu in the patient's blood between the two time points are predictive of the onset of clinical ovarian cancer.

66. A method of determining the presence of ovarian cancer in a patient, comprising determining levels of markers in a blood marker panel comprising three or more of EGF, G-CSF, IL-6, IL-8, VEGF and MCP-1 in as sample of the patient's blood, where the presence of three or more of the following conditions indicates the presence of ovarian cancer in the patient: EGF_{LO}, G-CSF_{HI}, IL-6_{HI}, IL-8_{HI}, VEGF_{HI} or MCP-1_{LO}.

67. The method of claim 66, wherein EGF_{LO} means less than about 224 pg/mL EGF, G-CSF_{HI} means greater than about 22 pg/mL G-CSF, IL-6_{HI} means greater than about 8.8 pg/mL IL-6, IL-8_{HI} means greater than about 10.2 pg/mL IL-8, CA-125_{HI}, means greater than about 10 pg/mL CA-125, VEGF_{HI} means greater than about 91 pg/mL VEGF or MCP-1_{LO} means less than about 342 pg/mL MCP-1.

68. The method of claim 66, comprising performing an immunoassay to determine the quantities of EGF, G-CSF, IL-6, IL-8, VEGF or MCP-1 in the patient's blood.

69. The method of claim 68, wherein the immunoassay utilizes an array comprising binding reagents types specific to EGF, G-CSF, IL-6, IL-8, VEGF and MCP-1, wherein each binding reagent type is attached independently to a one or more discrete locations on one or more surfaces of one or more substrates.

70. The method of claim 69, wherein the substrates are beads comprising an identifiable marker, wherein each binding reagent type is attached to a bead comprising a different identifiable marker than beads to which a different binding reagent type is attached.

71. The method of claim 70, wherein the identifiable marker comprises a fluorescent compound.

72. The method of claim 70, wherein the identifiable marker comprises a quantum dot.

73. An array comprising binding reagent types specific to any three or more of EGF, G-CSF, IL-6, IL-8, VEGF, CA-125 and MCP-1, wherein each binding reagent type is attached independently to one or more discrete locations on one or more surfaces of one or more substrates.

74. The array of claim 73, wherein the substrates are beads comprising an identifiable marker, wherein each binding reagent type is attached to a bead comprising a different identifiable marker than beads to which a different binding reagent type is attached.

75. The array of claim 74, wherein the identifiable marker comprises a fluorescent compound.

76. The array of claim 74, wherein the identifiable marker comprises a quantum dot.

77. The array of claim 73, further comprising a binding reagent type specific to CA-125 attached independently to one or more discrete locations, as compared to the other binding reagents, on one or more surfaces of the one or more substrates.
